SEQUENCE LISTING (10 sequences)

SEQ ID NO: 1 - DNA, 1125 bp, Homo Sapiens
aaatttcctttcactttcggtctctggctgtcacccggcttggccccttccacacccaactggggcaagcctgacccggcgacaggaggcatgaggggcccccggccgaaatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactgacctggccaaggcctcatcctgcggagccttaaacaacgcagtgagacagacatctatcatcccattttacaggggaggatactgaggcacacagaggggagtcaccagccagaggatgcatagcctggacacagaggaagttggctagaggccggtcccttccttgggcccctctcattccctccccagaatggaggcaacgccagaatccagcaccggccccatttacccaactctgtacaaagcccttgtccccatgaaattgtatataaatcatccttttctaccaaaaaaaaaaaaaaaaaaa

SEQ ID NO: 2 - protein, 235 aa, Homo Sapien
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 3 - DNA, 1042 bp, Homo sapien
tggggcaagcctgacccggcgacaggaggcatgaggggcccccggccgaaatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgccaggcctgatcctgttttctcccgcagtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactgacctggccaaggcctcatcctggggaggatactgaggcacacagaggggagtcaccagccagaggatgcatagcctggacacagaggaagttggctagaggccggtcccttccttgggcccctctcattccctccccagaatggaggcaacgccagaatccagcaccggccccatttacccaactctgtacaaagcccttgtccccatgaaattgtatataaatcatccttttctaccaaaaaaaaaaaaaaaaaaa

SEQ ID NO: 4 - protein, 153 aa, Homo sapien
MERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 5 - DNA, 678 bp, Homo sapien
ggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaa

SEQ ID NO: 6 - protein, 122 aa, Homo sapien
MKLLTGLVFCSLVLGVSSRSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 7 - DNA, 1719 bp, Homo sapien
gcggccgccctgctcctgctgcgtcgtgaggacccggggccgggggctggccccaggtaacccctccgcgtatgggaccgagctgggccaggtctcctggccgggccggggataccgtggggtatgcccagtgatgccagcagcttgtggcacctgggcgcaccctccagctcgggccccttccgatgggtctgctggctcaggtgcgggcgatggccggggagccgcgccccgcacgtgactcagcactttccccagagcccggactgcggagaacaatatcctcctccctaacagataaacagcccttgttcctcgggataaggactggcagtcccctgacaccctaagaccggcatctgtcgatgttatttccccagcatggccgaaacagaagccctgtcgaagcttcgggaagacttcaggatgcagaataaatccgtctttattttgggcgccagcggagaaaccggcagagtgctcttaaaggaaatcctggagcagggcctgttttccaaagtcacgctcattggccggaggaagctcaccttcgacgaggaagcttataaaaatgtgaatcaagaagtggtggactttgaaaagttggatgactacgcctctgcctttcaaggtcatgatgttggattctgttgcctgggtaccaccagagggaaagctggggcggagggatttgttcgtgttgaccgagattatgtgctgaagtctgcagagctggcaaaagctggagggtgcaaacatttcaacttgctatcctctaaaggagctgataaatcaagcaattttttatatctacaagttaagggagaagtagaagccaaggttgaagaattaaaatttgatcgttactctgtatttaggcctggagttctgttatgtgataggcaagaatctcgcccaggtgaatggctggttagaaagttctttggctccttaccagactcttgggccagtgggcattctgtgcctgtggtgaccgtggttagagcaatgctgaacaatgtggtgagaccaagagacaagcagatggaactgctggagaacaaggccatccatgacctggggaaagcgcatggctctctcaagccatgaccacattggagaaatggtttttattgtcaaccttaacacccatcaccaaatcggtaatttcagggtctaaaaaaagtcagcatgttttaactttgttgttttactatcctcaggcatccattccaatcaagaaatgatggtgctctgcatcagtggttcagagcctggttatacatatagatcactcagggagctttggaaaaataaagatttgtcagccctatctcaaacttgaatcaaaatttctggggtgtgggcacaataatctgtaattttctttgtttatacttcccctgatgccactggttccgatgccactggctggggggcctgctttgaaatgcttgtctgcagagtcacagcagccatgaaaaccttatgaccgtgcaaatgagctctgctctaaaattgttgacattcatgtctctgagttacaaaagtgctaattcactacatgtaattgtgtaagtaaacattgtgcctttactacttctttatgtaatagaagttatatacctaagcttatataatacatggggaggattaaataaaggaataaagatgaatggacaactcctaaaaaaaaaaaaaaaaaaaaaaaaaaaaaaa

SEQ ID NO: 8 - protein, 242 aa, Homo sapien
MAETEALSKLREDFRMQNKSVFILGASGETGRVLLKEILEQGLFSKVTLIGRRKLTFDEEAYKNVNQEVVDFEKLDDYASAFQGHDVGFCCLGTTRGKAGAEGFVRVDRDYVLKSAELAKAGGCKHFNLLSSKGADKSSNFLYLQVKGEVEAKVEELKFDRYSVFRPGVLLCDRQESRPGEWLVRKFFGSLPDSWASGHSVPVVTVVRAMLNNVVRPRDKQMELLENKAIHDLGKAHGSLKP

SEQ ID NO: 9 - DNA, 3220 bp, Homo sapien
aaacttttttccctggctctgccctgggtttccccttgaagggatttccctccgcctctgcaacaagaccctttataaagcacagactttctatttcactccgcggtatctgcatcgggcctcactggcttcaggagctgaataccctcccaggcacacacaggtgggacacaaataagggttttggaaccactattttctcatcacgacagcaacttaaaatgcctgggaagatggtcgtgatccttggagcctcaaatatactttggataatgtttgcagcttctcaagcttttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggatccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtctacatatcacccaagaatacagttatttctgtgaatccatccacaaagctgcaagaaggtggctctgtgaccatgacctgttccagcgagggtctaccagctccagagattttctggagtaagaaattagataatgggaatctacagcacctttctggaaatgcaactctcaccttaattgctatgaggatggaagattctggaatttatgtgtgtgaaggagttaatttgattgggaaaaacagaaaagaggtggaattaattgttcaagagaaaccatttactgttgagatctcccctggaccccggattgctgctcagattggagactcagtcatgttgacatgtagtgtcatgggctgtgaatccccatctttctcctggagaacccagatagacagccctctgagcgggaaggtgaggagtgaggggaccaattccacgctgaccctgagccctgtgagttttgagaacgaacactcttatctgtgcacagtgacttgtggacataagaaactggaaaagggaatccaggtggagctctactcattccctagagatccagaaatcgagatgagtggtggcctcgtgaatgggagctctgtcactgtaagctgcaaggttcctagcgtgtacccccttgaccggctggagattgaattacttaagggggagactattctggagaatatagagtttttggaggatacggatatgaaatctctagagaacaaaagtttggaaatgaccttcatccctaccattgaagatactggaaaagctcttgtttgtcaggctaagttacatattgatgacatggaattcgaacccaaacaaaggcagagtacgcaaacactttatgtcaatgttgcccccagagatacaaccgtcttggtcagcccttcctccatcctggaggaaggcagttctgtgaatatgacatgcttgagccagggctttcctgctccgaaaatcctgtggagcaggcagctccctaacggggagctacagcctctttctgagaatgcaactctcaccttaatttctacaaaaatggaagattctggggtttatttatgtgaaggaattaaccaggctggaagaagcagaaaggaagtggaattaattatccaagttactccaaaagacataaaacttacagcttttccttctgagagtgtcaaagaaggagacactgtcatcatctcttgtacatgtggaaatgttccagaaacatggataatcctgaagaaaaaagcggagacaggagacacagtactaaaatctatagatggcgcctataccatccgaaaggcccagttgaaggatgcgggagtatatgaatgtgaatctaaaaacaaagttggctcacaattaagaagtttaacacttgatgttcaaggaagagaaaacaacaaagactatttttctcctgagcttctcgtgctctattttgcatcctccttaataatacctgccattggaatgataatttactttgcaagaaaagccaacatgaaggggtcatatagtcttgtagaagcacagaagtcaaaagtgtagctaatgcttgatatgttcaactggagacactatttatctgtgcaaatccttgatactgctcatcattccttgagaaaaacaatgagctgagaggcagacttccctgaatgtattgaacttggaaagaaatgcccatctatgtcccttgctgtgagcaagaagtcaaagtaaaacttgctgcctgaagaacagtaactgccatcaagatgagagaactggaggagttccttgatctgtatatacaataacataatttgtacatatgtaaaataaaattatgccatagcaagattgcttaaaatagcaacactctatatttagattgttaaaataactagtgttgcttggactattataatttaatgcatgttaggaaaatttcacattaatatttgctgacagctgacctttgtcatctttcttctattttattccctttcacaaaattttattcctatatagtttattgacaataatttcaggttttgtaaagatgccgggttttatatttttatagacaaataataagcaaagggagcactgggttgactttcaggtactaaatacctcaacctatggtataatggttgactgggtttctctgtatagtactggcatggtacggagatgtttcacgaagtttgttcatcagactcctgtgcaactttcccaatgtggcctaaaaatgcaacttctttttattttcttttgtaaatgtttaggtttttttgtatagtaaagtgataatttctggaattagaaaaaaaaaaaaaaaaaaa

SEQ ID NO: 10 - protein, 739 aa, Homo sapien
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

(19) United States
(12) Patent Application Publication
(10) Pub. No.: US 2015/0377906 A1
(43) Pub. Date: Dec. 31, 2015

(54) BIOMARKERS FOR RADIATION EXPOSURE

(71) Applicants: THE REGENTS OF THE UNIVERSITY OF CALIFORNIA, Oakland, CA (US); THE METHODIST HOSPITAL, Houston, TX (US)

(72) Inventors: Andrew J. Wyrobek, Walnut Creek, CA (US); Leif E. Peterson, Houston, TX (US); Antoine M. Snijders, Antioch, CA (US)

(73) Assignees: THE METHODIST HOSPITAL, Houston, TX (US); THE REGENTS OF THE UNIVERSITY OF CALIFORNIA, Oakland, CA (US)

(21) Appl. No.: 14/752,282

(22) Filed: Jun. 26, 2015

Related U.S. Application Data
(60) Provisional application No. 62/018,501, filed on Jun. 27, 2014.

(51) Int. Cl. G01N 33/68; G01T 1/04; G01N 21/00

(52) U.S. Cl. CPC G01N 33/6893; G01N 21/00; G01T 1/04; G01N 2800/40; G01N 2800/60

(57) ABSTRACT

Panels of 1-, 2-, 3-, and 4 protein-biomarker for diagnostic and prognostic methods to determine a subject's radiation exposure and discriminates between persons who have been exposed to various levels of radiation, 1-5 days after exposure.

### CROSS-REFERENCE TO RELATED APPLICATIONS

This application priority to U.S. Provisional Patent Application No. 62/018,501, filed on Jun. 27, 2014. This application is related to U.S. Provisional Patent Application No. 61/901,372; and U.S. patent application Ser. No. 14/023,968, which claims priority to U.S. Provisional Patent Application No. 61/801,372, filed on Mar. 15, 2013 and to U.S. Provision 61/699,418, filed on Sep. 11, 2012, the contents of all of which are incorporated by reference in their entirety.

### STATEMENT OF GOVERNMENTAL SUPPORT

The invention was made with government support under Contract No. DE-AC02-05CH11231 awarded by the U.S. Department of Energy and under Contract No. HHSO100201000006C awarded by the Biomedical Advanced Research and Development Authority, Office of the Assistant Secretary for Preparedness and Response, Office of the Secretary, Department of Health and Human Services. The government has certain rights in the invention.

### BACKGROUND OF THE INVENTION

1. Field of the Invention

The invention relates to the field of diagnostic and prognostic methods using gene and protein biomarkers to determine a subject's radiation exposure and discriminating between persons who have been exposed to radiation only, and various levels of radiation exposure.

2. Related Art

Ionizing radiation causes well understood molecular, cellular, and tissue damage, with a broad range of severity in health effects that can range from non-detectable to acute radiation sickness and possibly, death. Exposure dose is the main predictor of the severity of the health effect. Unlike for medical procedures, assessing radiation dose in cases of nuclear accidents and nuclear terrorism remains a major unmet challenge. Rapid methods are needed to determine an individual's dose using small biological samples to separate the larger numbers of worried well from those individuals who will benefit most from immediate medical care. More than 200 mammalian proteins have been reported to be responsive to ionizing radiation [1], and currently there are no small panels of proteins capable of or for determining individual biodosimetry. Currently, there is no accepted method or protocol for human radiation biodosimetry, i.e., radiation exposure in cases where a physical dosimeter, such as a badge reader, is not available or practical.

Ossetrova and Blakely (2009) investigated the utility of multiple blood protein biomarkers for early-response assessment of radiation exposure using BALB/c mice. Serum amyloid A (SAA) was measured in plasma of irradiated mice using ELISA at 4, 24, 48 and 72 hr after whole body exposure to 0, 1, 2, 3.5, 5 and 7 Gy. Results showed significant dose-related increases in protein levels in plasma of irradiated mice. SAA was significantly increased at doses of 2 Gy and above at 24 hr only. This study was performed using an optimized ELISA protocol; their measurements were well above the LOD detection limit. The authors demonstrated that the use of multivariate discriminant analysis enhanced dose-dependent separation of irradiated animals from controls as the number of biomarkers increased.

Rithidech et al (2009) utilized two-dimensional electrophoresis gel coupled with mass spectrometry to analyze plasma proteins in CBA/CaJ mice exposed to 0 or 3 Gy. Plasma was collected from total body irradiated mice at 2 and 7 days post-irradiation. A dose dependent increase in both CC3 and VCAM levels was observed.

Prat (2005 and 2006) demonstrated that FLT3LG levels in the plasma of BALB/c mice were increased after whole body exposure to 2, 4, 7.5 and 11 Gy. Results showed that FLT3LG levels remained increased throughout the duration of the experiment which concluded at 28 days post irradiation.

Sugimoto (2001) demonstrated that C3H/HeN mice that were total body irradiated with 15 Gy showed a significant increase in serum FLT3LG levels.

### SUMMARY OF THE INVENTION

This investigation was undertaken to evaluate classification analysis of gold standard ELISA proteomic data for four candidate markers for individual radiation dose prediction. We are down-selecting candidate proteins to identify small panels of proteins for individual biodosimetry, using a mouse model as described herein and in Kim, D., Marchetti, F., Chen, Z., Zaric, S., Wilson, R. J., Hall, D. A., Gaster, R. S., Lee, J. R., Wang, J., Osterfeld, S. J., Yu, H., White, R. M., Blakely, W. F., Peterson, L. E., Bhatnagar, S., Mannion, B., Tseng, S., Roth, K., Coleman, M. A., Snijders, A. M., Wyrobek, A. J., Wang, S. X. Nanosensor dosimetry of mouse blood proteins after exposure to ionizing radiation. Scientific Reports(Nature). 3:2234, 2013, hereby incorporated by reference in its entirety.

We selected the following four biomarkers for analysis by ELISA: 1) FLT3 ligand (Fms-related tyrosine kinase 3 ligand; FLT3LG), which is a hematopoietic growth factor and is used as a clinical indicator for bone marrow status; 2) Serum amyloid A (SAA1), which is a major acute phase protein that is expressed and regulated in response to tissue injury and inflammation; 3) CC3, also known as HTATIP2 (HIV-1 Tat interactive protein 2), which is an oxidoreductase with proapoptotic as well as antiangiogenic properties; and 4) VCAM-1 (vascular cell adhesion molecule 1), which plays a role in cell-cell recognition [3].

Using an in vivo mouse radiation model, we developed protocols for measuring FLT3 ligand (FLT3LG), serum amyloid Al (SAA1), HIV-1 Tat interactive protein 2 (CC3) and vascular cell adhesion molecule 1 (VCAM-1) in small amounts of blood collected during the first week after X-ray exposures of sham, 0.1, 1, 2, 3, or 6 Gy. FLT3LG concentrations showed excellent dose discrimination at ≧1 Gy in the time window of 1 to 7 days after exposure except 1 Gy at day 7. SAA1 dose response was limited to the first two days after exposure. A multiplex assay with both and all four proteins showed improved dose classification accuracy.

Random forests analysis were then used for calculating permutation-based importance scores, feature selection frequency counts during decision tree learning, and for generating unsupervised and supervised cluster representations of samples via eigenanalysis of decision tree-based proximity matrices. We also employed several feature filtering and selection techniques, and a variety of supervised classification methods for class prediction of irradiation dose category.

Thus, the invention provides for a panel of one, two, three or four blood plasma proteins that are responsive to whole body radiation exposure and the development of an algorithm based on protein expression that discriminates individuals into 5 radiation-exposure categories: those not exposed to ionizing radiation, those exposed to 1Gy of radiation, or 2Gy of radiation, or 3Gy of radiation, or 6 Gy of radiation. Studies were conducted in mice and are described herein. Studies will be validated in human blood. Classification analysis resulted in assigning unirradiated and irradiated mice to their correct dose groups (0, 1, 2, 3, and 6 Gy) with 90-100% accuracy on day 1 and with 100% accuracy on day 5 after exposure.

### BRIEF DESCRIPTION OF THE FIGURES

FIGS. 1A and 1B. ELISA results for mouse FLT-3LG in Study 1 samples at 24 hr (FIG. 1A) and 5 days (FIG. 1B). Each blue triangle represents a different mouse. The red triangle represents the mean of the group. Sample dilutions ranged from 1:1 for sham to 1:10 for 6 Gy samples at 5 days. Samples from five mice were not analyzed because we did not have enough plasma to conduct multivariate analyses. Mean FLT-3LG concentrations (pg/mL±SD) for 0, 1, 2, 3, and 6 Gy were: 294±53, 445±48; 596±83, 724±94 and 913±126 at 24 hr; and 256±22, 419±44, 652±82, 1427±260 and 3747±779 at 5 days. All values for irradiated samples were statistically different from sham (see results). At 24 hr, four of the 6 Gy samples had absorbance levels near 1.0. Removing these samples, mean FLT-3LG concentration changes from 913±126 to 856±103 pg/mL, which is still highly statistically different from sham (P<2.1E-6; T-Test)

FIGS. 2A and 2B. ELISA results for mouse SAA1 in Study 1 samples at 24 hr (FIG. 2A) and 5 days (FIG. 2B). Each blue triangle represents a different mouse. The red triangle represents the mean of the group. Sample dilutions ranged from 1:500 for sham to 1:4000 for 6 Gy samples at 24 hr. Mean mouse SAA1 concentrations (μg/mL±SD) for 0, 1, 2, 3, and 6 Gy were: 100±20, 251±89; 617±119, 655±147 and 695±151 at 24 hr; and 70±10, 73±9, 71±21, 71±13 and 63±10 at 5 days. At 24 hr, all values for irradiated samples were statistically different from sham (see results). No difference with respect to sham was seen at 5 days. The data for 6 Gy at 5 days does not include two outlier mice that had SAA1 values of 1210 and 1760 μg/mL, respectively. Including these mice, mean SAA1 concentration changes from 63±10 to 258±543 μg/mL, which represent a 3.6-fold increase with respect to sham (P=0.09).

FIGS. 3A and 3B. ELISA results for mouse CC3 in Study 1 samples at 24 hr (FIG. 3A) and 5 days (FIG. 3B). Each blue triangle represents a different mouse. The red triangle represents the mean of the group. Sample dilutions ranged from 1:25000 for 5 day samples to 1:40000 for 24 hr samples. Results for the quality control are not shown. Mean CC3 concentrations (μg/mL±SD) for 0, 1, 2, 3, and 6 Gy were: 680±109, 773±138; 849±169, 1094±256 and 981±204 at 24 hr; and 592±149, 668±157, 608±121, 663±157 and 930±500 at 5 days. At 24 hr, all values for irradiated samples were statistically different from sham (see results). No difference with respect to sham was seen at 5 days. The data for 6 Gy at 5 days includes the mice that had CC3 value of 2305 μg/mL. Removing this mouse, CC3 mean concentration changes from 930±500 to 787±196 μg/mL. Interestingly, the two mice with the highest levels of CC3 at 6 Gy/5 days, are the same two outlier mice for mSAA1 levels.

FIGS. 4A and 4B. ELISA results for mouse VCAM in Study 1 samples at 24 hr (FIG. 4A) and 5 days (FIG. 4B). Each blue triangle represents a different mouse. The red triangle represents the mean of the group. All samples were diluted 1:400. Results for the quality control are not shown. Mean VCAM concentrations (μg/mL±SD) for 0, 1, 2, 3, and 6 Gy were: 772±228, 690±187; 635±183, 622±108 and 567±129 at 24 hr; and 883±237, 773±225, 712±219, 692±170 and 436±143 at 5 days. At 24 hr, decreases in VCAM levels were statistically significant with respect to sham for 3 (P<0.018) and 6 Gy (P<0.004); while at 5 days, they were statistically significant for 2 (P<0.042), 3 (P<0.013) and 6 Gy (P<0.0001).

FIGS. 5A, 5B, 5C, and 5D. Mouse ELISA standard curves for plasma biomarkers. FIG. 5A shows an example of the mouse FLT3LG standard curve produced using the R&D mFLT3LG ELISA kit. FIG. 5B shows an example of the mouse SAA1 standard curve produced using the Alpco SAA1 ELISA kit, which was used to analyze mouse plasma samples. FIG. 5C shows an example of the mouse CC3 standard curve produced using the Alpha Diagnostic mCC3 ELISA kit. The kit provides 5 standard concentrations already diluted and further dilution of these standards is not recommended. Indeed, dilution of these standards results in non-linear standard curves. FIG. 5D shows an example of the mouse VCAM standard curve produced using the Abnova mVCAM ELISA kit. Error bars indicate standard deviation between replicates.

FIG. 6. mFLT-3LG Dose response curves for male and female mice at day 1 and 5 after radiation, with confidence interval. Dose response curves with cubic fit and confidence interval represented by SD. Top three panels represent relative responses in male and female mice on day 1 after irradiation as well as the composite graph on the left. Bottom three panels represent day 5 results. X-axis is dose in Gy. Y axis is adjusted response (see text) (similar plots were prepared for SAA1, CC3, and VCAM, but not shown).

FIG. 7. Multivariate (FLT3LG, SAA1, CC3, VCAM) linear discriminant analysis results. Plots of mean distance (s.d.) from scores for zero-dose samples in canonical score space.

FIGS. 8A, 8B, 8C, and 8D. Mouse ELISA calibration curves for mouse plasma biomarkers. FIG. 8A shows the FLT3LG ELISA calibration curve in the absorbance range 0.05 A-0.7 A for 6 plates and 8 standards. Linear fits of the measured absorbance vs. true concentration of standards resulted in R2>0.99 for all plates. Points shown on the plot are the mean absorbance values (y-axis) for each true standard concentration (x-axis). By using dilution factors, the majority of mouse ELISA responses fell within the absorbance range shown. FIG. 8B shows the SAA1 ELISA calibration curve in the absorbance range 0.05 A-1 A for 6 plates and 5 standards. Linear fits of the measured absorbance vs. true concentration of standards resulted in R2>0.99 for all plates. Points shown on the plot are the mean absorbance values (y-axis) for each true standard concentration (x-axis). By using dilution factors, the majority of mouse ELISA responses fell within the absorbance range shown. FIG. 8C shows CC3 ELISA calibration curve in the absorbance range 0.3 A-1.8 A for 6 plates and 5 standards. Linear fits of the measured absorbance vs. true concentration of standards resulted in R2>0.97 for all plates. Points shown on the plot are the mean absorbance values (y-axis) for each true standard concentration (x-axis). By using dilution factors, the majority of mouse ELISA responses fell within the absorbance range shown. FIG. 8D shows the VCAM ELISA calibration curve in the absorbance range 0.1 A-1.8 A for 5 plates and 9 standards. Linear fits of the measured absorbance vs. true concentration of standards resulted in R2>0.98 for all plates. Points shown on the plot are the mean absorbance values (y-axis) for each true standard concentration (x-axis). By using dilution factors, the majority of mouse ELISA responses fell within the absorbance range shown.

FIGS. 9A, 9B, 10A, and 10B. Histogram plots of ELISA replicate values for mouse plasma biomarkers ELISA data before (left panel) and after removing skew and mean-zero standardization (right panel) is shown. Note: the x-axis variable named “zlnflt31g” in the right panel represents Z-scores (mean-zero standardized) of loge-transformed FLT3LG (FIG. 9A) concentration from the calibration curve. The same is true for SAA1 (FIG. 9B), CC3 (FIG. 10A) and VCAM (FIG. 10B).

FIG. 11. Random forest (RF) feature informativeness for all data (day 1 and day 5 post-exposure). Top panel: Importance scores for all features based on 10,000 trees. Day 1 and Day 5 data combined. Middle panel: Frequency of selection of each feature for first node splits. Bottom panel: Frequency of selection of each feature for all node splits.

FIG. 12. Random forest (RF) biomarker informativeness for irradiation response at day 1 post-exposure. Top panel: Importance scores for biomarker response at Day 1 using 1,000 trees. Middle panel: Frequency of selection of biomarkers for first node splits. Bottom panel: Frequency of selection of biomarkers for all node splits.

FIG. 13. Random forest (RF) biomarker informativeness for irradiation response at day 5 post-exposure. Top panel: Importance scores for biomarker response at Day 5 using 1,000 trees. Middle panel: Frequency of selection of biomarkers for first node splits. Bottom panel: Frequency of selection of biomarkers for all node splits.

FIG. 14. Random forest (RF) supervised (top panel) and unsupervised (bottom panel) cluster analysis based on 2D principal component score plots for day-1 input features using random forest proximity matrix for 1,000 trees.

FIG. 15. Random forest (RF) supervised (top panel) and unsupervised (bottom panel) cluster analysis based on 2D principal component score plots for day-5 input features using random forest proximity matrix for 1,000 trees.

Table 1. Number of class comparisons, M, and number of biomarkers, Nₘ, filtered for each comparison.

Table 2. Supervised classification accuracy for 5-class problem involving dose categories 0 Gy, 1 Gy, 2 Gy, 3 Gy, and 6 Gy for Day-1 ELISA responses. Leave-one-out cross validation used.

Table 3. Classification accuracy for 5-class problem involving dose categories 0 Gy, 1 Gy, 2 Gy, 3 Gy, and 6 Gy for Day-5 ELISA responses. Leave-one-out cross validation used.

Tables 4A, 4B, 4C, and 4D. Gender variation and dose dependence of plasma protein concentrations with time after exposures. Dose response tables for each biomarker-average concentrations (all samples), average concentrations (males and females), standard deviation; separated by time points (24 hr and 5 d). (Table 4A) Ht-31g. (Table 4B) SAA1. (Table 4C) CC3. (Table 4D) VCAM1.

Table 5. Receiver operator characteristic (ROC) curve are under the curve (AUC) for 2 class models using k-nearest neighbors (k=7, “7NN”) with leave-one-out cross validation (LOOCV) and linear discriminant analysis (LDA) for Day 1 and Day 5.

Table 6. Receiver operator characteristic (ROC) area under the curve (AUC) for one-against-other classification analyses for triplicate ELISA Day 1 and 5 response using k-nearest neighbor (k=7, i.e. “7NN”) and linear discriminant analysis (LDA) with leave-one-out cross validation (LOOCV) for Day 1 and Day 5.

Tables 7A, 7B, and 7C. (Table 7A) T-test results (t-statistics and p-values in parentheses) for FLT3LG concentrations between two doses on day 1 (above diagonal line) and day 5 (below diagonal line). Conclusion: FLT3LG mean concentration in plasma was significantly different between all pairs of doses on day 1 and day 5 after irradiation. (Table 7B) T-test results (t-statistics and p-values in parentheses) for FLT3LG concentrations between two dose groups on day 1. Conclusion: t-test results show that on day 1 after irradiation, FLT3LG concentration in plasma was significantly different between 0 GY and groups (1 Gy, 2 Gy), (1 Gy, 2 Gy, 3 Gy), (1 Gy, 2 Gy, 3 Gy, 6 Gy, between 1 Gy and groups (2 Gy, 3Gy), (2 Gy, 3 Gy, 6Gy), between 2 Gy and group (3 Gy, 6 Gy). (Table 7C) T-test results (t-statitics and p-values in parentheses) for FLT3LG concentrations between two dose groups on day 5. Conclusion: t-test results indicate that on day 5 after irradiation, FLT3LG concentration in plasma was significantly different between 0 GY and groups (1 Gy, 2 Gy), (1 Gy, 2 Gy, 3 Gy), (1 Gy, 2 Gy, 3 Gy, 6 Gy), between 1 Gy and groups (2 Gy, 3Gy), (2 Gy, 3 Gy, 6Gy), between 2 Gy and group (3 Gy, 6 Gy).

Tables 8A, 8B, and 8C. (Table 8A) T-test results (t-statistics and p-values in parentheses) for SAA1 concentrations between two doses on 1-day (above diagonal line) and 5-day (below diagonal line). Conclusion: SAA1 mean concentration in plasma was significantly different between 0 Gy, 2 Gy, 3 Gy, 6 Gy and between 1 Gy and 2 Gy, 3Gy, 6 Gy but not between 2 Gy and 3 Gy, 6 Gy and also not between 3 Gy and 6 Gy on day 1 after irradiation. On day 5, SAA1 mean concentration was not significantly different between any two doses. (Table 8B). T-test results (t-statistics and p-values in parentheses) for SAA1 concentrations between two dose groups on day 1. Conclusion: t-test results show that on day 1 after irradiation, SAA1 concentration in plasma was significantly different between 0 GY and groups (1 Gy, 2 Gy), (1 Gy, 2 Gy, 3 Gy), (1 Gy, 2 Gy, 3 Gy, 6 Gy), between 1 Gy and groups (2 Gy, 3Gy), (2 Gy, 3 Gy, 6Gy), but not between 2 Gy and group (3 Gy, 6 Gy). (Table 8C) T-test results (t-statistics and p-values in parentheses) for SAA1 concentrations between two dose groups on day 5. Conclusion: SAA1 concentration in plasma was not significantly different between these dose groups on day 5 after irradiation.

Tables 9A, 9B, and 9C. (Table 9A) T-test results (t-statistics and p-values in parentheses) for CC3 concentrations between two doses on day 1 (above diagonal line) and day 5 (below diagonal line). Conclusion: t-test results indicate that CC3 concentration in plasma was significantly different between 0 Gy and 2 Gy, 3 Gy, 6 Gy, and between 1 Gy and 3Gy, 6 Gy on day 1 after irradiation; on day 5, CC3 concentration was significantly different between 0 Gy and 6 Gy and between 2 Gy and 6 Gy. (Table 9B) T-test results (t-statistics and p-values in parentheses) for CC3 concentrations between two dose groups on day 1. Conclusion: T-test results indicate significantly different mean concentration between 0 GY and groups (1 Gy, 2 Gy), (1 Gy, 2 Gy, 3 Gy), (1 Gy, 2 Gy, 3 Gy, 6 Gy), between 1 Gy and groups (2 Gy, 3 Gy, 6Gy), between 2 Gy and group (3 Gy, 6 Gy) on day 1. (Table 9C) T-test results (t-statistics and p-values in parentheses) for CC3 concentrations between two dose groups on day 5. Conclusion: During 5 days after irradiation, CC3 concentration in plasma was not significantly different between 0 GY and groups (1 Gy, 2 Gy), (1 Gy, 2 Gy, 3 Gy), (1 Gy, 2 Gy, 3 Gy, 6 Gy, between 1 Gy and groups (2 Gy, 3 Gy, 6Gy), between 2 Gy and group (3 Gy, 6 Gy).

Tables 10A, 10B, and 10C. (Table 10A) T-test results (t-statistics and p-values in parentheses) for VCAM concentrations between two doses on day 1 (above diagonal line) and day 5 (below diagonal line). Conclusion: on day 1 after irradiation, VCAM concentration in plasma was significantly different between 0 Gy and 3 Gy, 6 Gy, and between 1 Gy and 6 Gy and on day 5, was very significantly different between 6 Gy and 0 Gy, 2 Gy, 3 Gy. (Table 10B) T-test results (t-statistics and p-values in parentheses) for VCAM concentrations between two dose groups on day 1. Conclusion: 0 GY and groups (1 Gy, 2 Gy, 3 Gy), (1 Gy, 2 Gy, 3 Gy, 6 Gy) have significant differences for VCAM concentration in plasma on day 1 after irradiation. (Table 10C) T-test results (t-statistics and p-values in parentheses) for VCAM concentrations between two dose groups on day 5. Conclusion: 0 GY and groups (1 Gy, 2 Gy, 3 Gy), (1 Gy, 2 Gy, 3 Gy, 6 Gy), 1 Gy and group (2 Gy, 3 Gy, 6 Gy) have significant differences for VCAM concentration in plasma on day 5 after irradiation.

### DESCRIPTION OF THE PREFERRED EMBODIMENTS

Herein are described systems, methods and compositions for the identification of a panel of genes whose gene expression and protein levels in part provide a signature for determining individual radiation dose prediction and dosimetry. In some embodiments, 1-gene, 2-gene, 3-gene, or 4-gene blood protein biomarker panel signature is described. In other embodiments, ELISA-based methods for analyzing irradiation responses of proteomic biomarkers for dose category prediction.

In various embodiments, 1-, 2-, 3- and 4-identified blood proteins are sufficient to provide a 100% classification accuracy for assigning individuals to the correct radiation exposure dose (5 class problem). Further development of this blood protein panel is to test the same proteins in blood from irradiated persons or in irradiated blood cells collected from healthy normal people. We have already demonstrated the efficacy of the latter approach to develop a biodosimetry panel for human blood. We previously used human blood exposed ex vivo to ionizing radiation to develop a panel of blood biomarkers consisting of a combination of several blood mRNAs and proteins. Blood proteins are more stable than blood mRNA, and therefore more promising for biodosimetry and were the focus of the present Examples. The high level of homology between genes/proteins of mice and humans makes the in vivo mouse model extremely suitable for biomarker-discovery studies (Rithidech 2009).

Thus, the present panels can also be used to determine an individual's degree of radiation exposure from 1, 2, 3, 4, to 5 days after such exposure. In some embodiments, the panel of four blood plasma proteins that are responsive to whole body radiation exposure and the methods of analysis of protein expression, whereby the result of the analysis provides for patient discrimination and selection that discriminates individuals into 5 radiation-exposure categories: those not exposed to ionizing radiation, those exposed to 1Gy of radiation, or 2Gy of radiation, or 3Gy of radiation, or 6 Gy of radiation.

In various embodiments, the measurement and detection of blood protein levels are from a sample from a patient. In some embodiments, the protein levels are analyzed and determined by Enzyme-Linked Immunosorbant Assay (ELISA). Such methods for protein analyses are well known to those skilled in the art. Suitable methods for ELISAs are described in Kim, D., Marchetti, F., Chen, Z., Zaric, S., Wilson, R. J., Hall, D. A., Gaster, R. S., Lee, J. R., Wang, J., Osterfeld, S. J., Yu, H., White, R. M., Blakely, W. F., Peterson, L. E., Bhatnagar, S., Mannion, B., Tseng, S., Roth, K., Coleman, M. A., Snijders, A. M., Wyrobek, A. J., Wang, S. X. Nanosensor dosimetry of mouse blood proteins after exposure to ionizing radiation. Scientific Reports(Nature). 3:2234, 2013, and Budworth, H., Snijders, A. M., Marchetti, F., Mannion, B., Bhatnagar, S., Kwoh, E., Tan, Y., Wang, S. X., Blakely, W. F., Coleman, M. A., Peterson, L. E., Wyrobek, A. J. DNA repair and cell cycle biomarkers of radiation exposure and inflammation stress in human blood. PLoS One. 7(11):e48619, 2012, both of which are hereby incorporated by reference in their entirety.

We selected the following four biomarkers for analysis by ELISA: 1) FLT3 ligand (Fms-related tyrosine kinase 3 ligand; FLT3LG), which is a hematopoietic growth factor and is used as a clinical indicator for bone marrow status; 2) Serum amyloid A (SAA1), which is a major acute phase protein that is expressed and regulated in response to tissue injury and inflammation; 3) CC3, also known as HTATIP2 (HIV-1 Tat interactive protein 2), which is an oxidoreductase with proapoptotic as well as antiangiogenic properties; and 4) VCAM-1 (vascular cell adhesion molecule 1), which plays a role in cell-cell recognition [3].

Methods for detection of expression levels of a gene can be carried out using known methods in the art including but not limited to, optical density, fluorescent intensity, fluorescent in situ hybridization (FISH), immunohistochemical analysis, fluorescence detection, comparative genomic hybridization, PCR methods including real-time and quantitative PCR, mass and imaging spectrometry and spectroscopy methods and other sequencing and analysis methods known or developed in the art. The expression level of the gene in question can be measured by measuring the amount or number of molecules of mRNA or transcript in a cell. The measuring can comprise directly measuring the mRNA or transcript obtained from a cell, or measuring the cDNA obtained from an mRNA preparation thereof. Such methods of extracting the mRNA or transcript from a cell, or preparing the cDNA thereof are well known to those skilled in the art. In other embodiments, the expression level of a gene can be measured by measuring or detecting the amount of protein or polypeptide expressed, such as measuring the amount of antibody that specifically binds to the protein in a dot blot or Western blot. The proteins described in the present invention can be overexpressed and purified or isolated to homogeneity and antibodies raised that specifically bind to each protein. Such methods are well known to those skilled in the art.

Comparison of the detected expression level of a gene in a patient sample is often compared to the expression levels detected in a normal tissue sample or a reference expression level. In some embodiments, the reference expression level can be the average or normalized expression level of the gene or gene product in a known panel of standards or a panel of normal cell lines or cancer cell lines.

In various embodiments, the expression levels of proteins in the biomarker panel of FLT3LG, SAA1, CC3, and/or VCAM, are measured and analyzed to determine an individual subject's radiation exposure level , comprising: (a) measuring the protein expression level of the blood proteins FLT3LG, SAA1, CC3, and/or VCAM, each protein in one of the biomarker panels in a sample from a patient, whereby based on the calculated methods and analyses a patient prediction score is used to determine the level of radiation exposure of a patient.

Gene sequences and gene products that may be detected are herein identified by gene name, Unigene ID, GeneID and/or GenBank Accession Numbers, and the publicly available content all of which are hereby incorporated by reference in their entireties for all purposes. As understood in the art, there are naturally occurring polymorphisms for many gene sequences. Genes that are naturally occurring allelic variations for the purposes of this invention are those genes encoded by the same genetic locus. The proteins which are detected and encoded by allelic variations of the four proteins FLT3LG, SAA1, CC3, and/or VCAM typically have at least 95% amino acid sequence identity to one another, i.e., an allelic variant of a gene indicated in herein typically encodes a protein product that has at least 95% identity, often at least 96%, at least 97%, at least 98%, or at least 99%, or greater, identity to the amino acid sequence encoded by the nucleotide sequence denoted by the Entrez Gene ID number (as of Jun. 25, 2014) shown herein for that gene. For example, an allelic variant of a gene encoding FLT3LG (gene: Homo sapiens fms-related tyrosine kinase 3 ligand (FLT3LG)) typically has at least 95% identity, often at least 96%, at least 97%, at least 98%, or at least 99%, or greater, to the FLT3LG protein sequence encoded by the nucleic acid sequence available under Entrez Gene ID no. 2323). In some cases, a “gene identified in” herein, may also refer to an isolated polynucleotide that can be unambiguously mapped to the same genetic locus as that of a gene assigned to a genetic locus by the Entrez Gene ID or it may also refer to an expression product that is encoded by a polynucleotide that can be unambiguously mapped to the same genetic locus as that of a gene assigned to a genetic locus by the Entrez Gene ID.

FLT3LG: Homo sapiens fms-related tyrosine kinase 3 ligand (FLT3LG) Nucleotide sequence GenBank Accession No. NM_{—}001204502.1 GI:325197196, encodes FLT3LG protein sequence GenBank Accession No. NP_{—}001191431.1 GI:325197197, both of which are hereby incorporated by reference.

Dendritic cells (DCs) provide the key link between innate and adaptive immunity by recognizing pathogens and priming pathogen-specific immune responses. FLT3LG controls the development of DCs and is particularly important for plasmacytoid DCs and CD8 (see MIM 186910)-positive classical DCs and their CD103 (ITGAE; MIM604682)-positive tissue counterparts (summary by Sathaliyawala et al., 2010 [PubMed 20933441]).[supplied by OMIM, January 2011]. SEQ ID NO: 1 is the FLT3LG nucleotide sequence and Transcript Variant isoform 1. This variant (1) encodes the longer isoform (1). Variants 1, 2, and 3 encode the same isoform (1).

```
(SEQ ID No: 1)
      1 aaatttcctt tcactttcgg tctctggctg tcacccggct tggccccttc cacacccaac
    61 tggggcaagc ctgacccggc gacaggaggc atgaggggcc cccggccgaa atgacagtgc
  121 tggcgccagc ctggagccca acaacctatc tcctcctgct gctgctgctg agctcgggac
  181 tcagtgggac ccaggactgc tccttccaac acagccccat ctcctccgac ttcgctgtca
  241 aaatccgtga gctgtctgac tacctgcttc aagattaccc agtcaccgtg gcctccaacc
  301 tgcaggacga ggagctctgc gggggcctct ggcggctggt cctggcacag cgctggatgg
  361 agcggctcaa gactgtcgct gggtccaaga tgcaaggctt gctggagcgc gtgaacacgg
  421 agatacactt tgtcaccaaa tgtgcctttc agcccccccc cagctgtctt cgcttcgtcc
  481 agaccaacat ctcccgcctc ctgcaggaga cctccgagca gctggtggcg ctgaagccct
  541 ggatcactcg ccagaacttc tcccggtgcc tggagctgca gtgtcagccc gactcctcaa
  601 ccctgccacc cccatggagt ccccggcccc tggaggccac agccccgaca gccccgcagc
  661 cccctctgct cctcctactg ctgctgcccg tgggcctcct gctgctggcc gctgcctggt
  721 gcctgcactg gcagaggacg cggcggagga caccccgccc tggggagcag gtgccccccg
  781 tccccagtcc ccaggacctg ctgcttgtgg agcactgacc tggccaaggc ctcatcctgc
  841 ggagccttaa acaacgcagt gagacagaca tctatcatcc cattttacag gggaggatac
  901 tgaggcacac agaggggagt caccagccag aggatgcata gcctggacac agaggaagtt
  961 ggctagaggc cggtcccttc cttgggcccc tctcattccc tccccagaat ggaggcaacg
1021 ccagaatcca gcaccggccc catttaccca actctgtaca aagcccttgt ccccatgaaa
1081 ttgtatataa atcatccttt tctaccaaaa aaaaaaaaaa aaaaa
(SEQ ID No: 2)
      1 mtvlapawsp ttylllllll ssglsgtqdc sfqhspissd favkirelsd yllqdypvtv
    61 asnlqdeelc gglwrlvlaq rwmerlktva gskmqgller vnteihfvtk cafqpppscl
  121 rfvqtnisrl lqetseqlva lkpwitrqnf srclelqcqp dsstlpppws prpleatapt
  181 apqpplllll llpvglllla aawclhwqrt rrrtprpgeq vppvpspqdl llveh
NM_001278637.1 GI:520975468
Homo sapiens fms-related tyrosine kinase 3 ligand (FLT3LG), transcript
variant 4, mRNA
Transcript Variant: This variant (4) uses an alternate splice site in the 5′
UTR which results in the use of a downstream AUG compared to variant 1. The
encoded isoform (2) has a shorter N-terminus compared to isoform 1.
(SEQ ID No: 3)
      1 aaatttcctt tcactttcgg tctctggctg tcacccggct tggccccttc cacacccaac
    61 tggggcaagc ctgacccggc gacaggaggc atgaggggcc cccggccgaa atgacagtgc
  121 tggcgccagc ctggagccca acaacctatc tcctcctgct gctgctgctg agctcgggac
  181 tcagtgggac ccaggactgc tccttccaac acagccccat ctcctccgac ttcgctgtca
  241 aaatccgtga gctgccaggc ctgatcctgt tttctcccgc agtctgacta cctgcttcaa
  301 gattacccag tcaccgtggc ctccaacctg caggacgagg agctctgcgg gggcctctgg
  361 cggctggtcc tggcacagcg ctggatggag cggctcaaga ctgtcgctgg gtccaagatg
  421 caaggcttgc tggagcgcgt gaacacggag atacactttg tcaccaaatg tgcctttcag
  481 ccccccccca gctgtcttcg cttcgtccag accaacatct cccgcctcct gcaggagacc
  541 tccgagcagc tggtggcgct gaagccctgg atcactcgcc agaacttctc ccggtgcctg
  601 gagctgcagt gtcagcccga ctcctcaacc ctgccacccc catggagtcc ccggcccctg
  661 gaggccacag ccccgacagc cccgcagccc cctctgctcc tcctactgct gctgcccgtg
  721 ggcctcctgc tgctggccgc tgcctggtgc ctgcactggc agaggacgcg gcggaggaca
  781 ccccgccctg gggagcaggt gccccccgtc cccagtcccc aggacctgct gcttgtggag
  841 cactgacctg gccaaggcct catcctgggg aggatactga ggcacacaga ggggagtcac
  901 cagccagagg atgcatagcc tggacacaga ggaagttggc tagaggccgg tcccttcctt
  961 gggcccctct cattccctcc ccagaatgga ggcaacgcca gaatccagca ccggccccat
1021 ttacccaact ctgtacaaag cccttgtccc catgaaattg tatataaatc atccttttct
1081 accaaaaaaa aaaaaaaaaa aa
NP_001265566.1 GI:520975469
fms-related tyrosine kinase 3 ligand isoform 2 [Homo sapiens]
(SEQ ID No: 4)
      1 merlktvags kmqgllervn teihfvtkca fqpppsclrf vqtnisrllq etseqlvalk
    61 pwitrqnfsr clelqcqpds stlpppwspr pleataptap qpplllllll pvgllllaaa
  121 wclhwqrtrr rtprpgeqvp pvpspqdlll veh
```

SAA1: Homo sapiens serum amyloid A1 (SAA1). Nucleotide sequence GenBank Accession No. NM_{—}000331.4 GI:295821191 encodes SAA1 protein sequence GenBank Accession No. NP_{—}000322.2 GI:40316912, both of which are hereby incorporated by reference. This gene encodes a member of the serum amyloid A family of apolipoproteins. The encoded protein is a major acute phase protein that is highly expressed in response to inflammation and tissue injury. . It represents a family of low molecular weight acute phase proteins, which are produced primarily by the liver in response to infection and inflammatory stimuli (Glojnaric, 2007). This protein also plays an important role in HDL metabolism and cholesterol homeostasis. High levels of this protein are associated with chronic inflammatory diseases including atherosclerosis, rheumatoid arthritis, Alzheimer's disease and Crohn's disease. This protein may also be a potential biomarker for certain tumors. Alternate splicing results in multiple transcript variants that encode the same protein. A pseudogene of this gene is found on chromosome 11.[provided by RefSeq, Jun 2012]. This variant (1) represents the longest transcript. Variants 1, 2 and 3 encode the same protein.

```
NM_000331.4 GI:295821191
Homo sapiens serum amyloid A1 (SAA1), transcript variant 1, mRNA
(SEQ ID No: 5)
    1 ggcagggacc cgcagctcag ctacagcaca gatcaggtga ggagcacacc aaggagtgat
  61 ttttaaaact tactctgttt tctctttccc aacaagatta tcatttcctt taaaaaaaat
121 agttatcctg gggcatacag ccataccatt ctgaaggtgt cttatctcct ctgatctaga
181 gagcaccatg aagcttctca cgggcctggt tttctgctcc ttggtcctgg gtgtcagcag
241 ccgaagcttc ttttcgttcc ttggcgaggc ttttgatggg gctcgggaca tgtggagagc
301 ctactctgac atgagagaag ccaattacat cggctcagac aaatacttcc atgctcgggg
361 gaactatgat gctgccaaaa ggggacctgg gggtgcctgg gctgcagaag tgatcagcga
421 tgccagagag aatatccaga gattctttgg ccatggtgcg gaggactcgc tggctgatca
481 ggctgccaat gaatggggca ggagtggcaa agaccccaat cacttccgac ctgctggcct
541 gcctgagaaa tactgagctt cctcttcact ctgctctcag gagatctggc tgtgaggccc
601 tcagggcagg gatacaaagc ggggagaggg tacacaatgg gtatctaata aatacttaag
661 aggtggaatt tgtggaaa
NP_000322.2 GI:40316912
serum amyloid A-1 protein preproprotein [Homo sapiens]
(SEQ ID No: 6)
    1 mklltglvfc slvlgvssrs ffsflgeafd gardmwrays dmreanyigs dkyfhargny
  61 daakrgpgga waaevisdar eniqrffghg aedsladqaa newgrsgkdp nhfrpaglpe
121 ky
```

CC3: oxidoreductase HTATIP2 isoform b [Homo sapiens], HIV-1 TAT-interactive protein 2. Nucleotide sequence GenBank Accession No. NM_{—}001098522.1 GI:148728171 encodes the CC3 protein sequence GenBank Accession No. NP_{—}001091992.1 GI:148728172, both of which are hereby incorporated by reference. This variant (4) has an alternate 5′ end and differs in the 5′ UTR, compared to variant 1. These differences cause translation initiation at a downstream AUG and an isoform (b, also known as CC3) with a shorter N-terminus compared to isoform a. Variants 2, 3 and 4 encode the same isoform.

```
NM_001098522.1 GI:148728171
Homo sapiens HIV-1 Tat interactive protein 2, 30 kDa (HTATIP2),
transcript variant 4, mRNA
(SEQ ID No: 7)
      1 gcggccgccc tgctcctgct gcgtcgtgag gacccggggc cgggggctgg ccccaggtaa
    61 cccctccgcg tatgggaccg agctgggcca ggtctcctgg ccgggccggg gataccgtgg
  121 ggtatgccca gtgatgccag cagcttgtgg cacctgggcg caccctccag ctcgggcccc
  181 ttccgatggg tctgctggct caggtgcggg cgatggccgg ggagccgcgc cccgcacgtg
  241 actcagcact ttccccagag cccggactgc ggagaacaat atcctcctcc ctaacagata
  301 aacagccctt gttcctcggg ataaggactg gcagtcccct gacaccctaa gaccggcatc
  361 tgtcgatgtt atttccccag catggccgaa acagaagccc tgtcgaagct tcgggaagac
  421 ttcaggatgc agaataaatc cgtctttatt ttgggcgcca gcggagaaac cggcagagtg
  481 ctcttaaagg aaatcctgga gcagggcctg ttttccaaag tcacgctcat tggccggagg
  541 aagctcacct tcgacgagga agcttataaa aatgtgaatc aagaagtggt ggactttgaa
  601 aagttggatg actacgcctc tgcctttcaa ggtcatgatg ttggattctg ttgcctgggt
  661 accaccagag ggaaagctgg ggcggaggga tttgttcgtg ttgaccgaga ttatgtgctg
  721 aagtctgcag agctggcaaa agctggaggg tgcaaacatt tcaacttgct atcctctaaa
  781 ggagctgata aatcaagcaa ttttttatat ctacaagtta agggagaagt agaagccaag
  841 gttgaagaat taaaatttga tcgttactct gtatttaggc ctggagttct gttatgtgat
  901 aggcaagaat ctcgcccagg tgaatggctg gttagaaagt tctttggctc cttaccagac
  961 tcttgggcca gtgggcattc tgtgcctgtg gtgaccgtgg ttagagcaat gctgaacaat
1021 gtggtgagac caagagacaa gcagatggaa ctgctggaga acaaggccat ccatgacctg
1081 gggaaagcgc atggctctct caagccatga ccacattgga gaaatggttt ttattgtcaa
1141 ccttaacacc catcaccaaa tcggtaattt cagggtctaa aaaaagtcag catgttttaa
1201 ctttgttgtt ttactatcct caggcatcca ttccaatcaa gaaatgatgg tgctctgcat
1261 cagtggttca gagcctggtt atacatatag atcactcagg gagctttgga aaaataaaga
1321 tttgtcagcc ctatctcaaa cttgaatcaa aatttctggg gtgtgggcac aataatctgt
1381 aattttcttt gtttatactt cccctgatgc cactggttcc gatgccactg gctggggggc
1441 ctgctttgaa atgcttgtct gcagagtcac agcagccatg aaaaccttat gaccgtgcaa
1501 atgagctctg ctctaaaatt gttgacattc atgtctctga gttacaaaag tgctaattca
1561 ctacatgtaa ttgtgtaagt aaacattgtg cctttactac ttctttatgt aatagaagtt
1621 atatacctaa gcttatataa tacatgggga ggattaaata aaggaataaa gatgaatgga
1681 caactcctaa aaaaaaaaaa aaaaaaaaaa aaaaaaaaa
NP_001091992.1 GI:148728172
(SEQ ID No: 8)
      1 maetealskl redfrmqnks vfilgasget grvllkeile qglfskvtli grrkltfdee
    61 ayknvnqevv dfeklddyas afqghdvgfc clgttrgkag aegfvrvdrd yvlksaelak
  121 aggckhfnll sskgadkssn flylqvkgev eakveelkfd rysvfrpgvl lcdrqesrpg
  181 ewlvrkffgs lpdswasghs vpvvtvvram lnnvvrprdk qmellenkai hdlgkahgsl
  241 kp
```

VCAM-1 (also referred to herein as VCAM): Vascular cell adhesion molecule. Nucleotide GenBank Accession No. NM_{—}001078.3 GI:31543426 encodes protein GenBank Accession No. NP_{—}001069.1 GI:4507875 both of which are hereby incorporated by reference. This gene is a member of the Ig superfamily and encodes a cell surface sialoglycoprotein expressed by cytokine-activated endothelium. This type I membrane protein mediates leukocyte-endothelial cell adhesion and signal transduction, and may play a role in the development of artherosclerosis and rheumatoid arthritis. Three alternatively spliced transcripts encoding different isoforms have been described for this gene. [provided by RefSeq, December 2010]. This variant (1) encodes the predominant, full-length isoform (a).

```
Homo sapiens vascular cell adhesion molecule 1 (VCAM1), transcript
variant 1, mRNA
NM_001078.3 GI:31543426
(SEQ ID No: 9)
      1 aaactttttt ccctggctct gccctgggtt tccccttgaa gggatttccc tccgcctctg
    61 caacaagacc ctttataaag cacagacttt ctatttcact ccgcggtatc tgcatcgggc
  121 ctcactggct tcaggagctg aataccctcc caggcacaca caggtgggac acaaataagg
  181 gttttggaac cactattttc tcatcacgac agcaacttaa aatgcctggg aagatggtcg
  241 tgatccttgg agcctcaaat atactttgga taatgtttgc agcttctcaa gcttttaaaa
  301 tcgagaccac cccagaatct agatatcttg ctcagattgg tgactccgtc tcattgactt
  361 gcagcaccac aggctgtgag tccccatttt tctcttggag aacccagata gatagtccac
  421 tgaatgggaa ggtgacgaat gaggggacca catctacgct gacaatgaat cctgttagtt
  481 ttgggaacga acactcttac ctgtgcacag caacttgtga atctaggaaa ttggaaaaag
  541 gaatccaggt ggagatctac tcttttccta aggatccaga gattcatttg agtggccctc
  601 tggaggctgg gaagccgatc acagtcaagt gttcagttgc tgatgtatac ccatttgaca
  661 ggctggagat agacttactg aaaggagatc atctcatgaa gagtcaggaa tttctggagg
  721 atgcagacag gaagtccctg gaaaccaaga gtttggaagt aacctttact cctgtcattg
  781 aggatattgg aaaagttctt gtttgccgag ctaaattaca cattgatgaa atggattctg
  841 tgcccacagt aaggcaggct gtaaaagaat tgcaagtcta catatcaccc aagaatacag
  901 ttatttctgt gaatccatcc acaaagctgc aagaaggtgg ctctgtgacc atgacctgtt
  961 ccagcgaggg tctaccagct ccagagattt tctggagtaa gaaattagat aatgggaatc
1021 tacagcacct ttctggaaat gcaactctca ccttaattgc tatgaggatg gaagattctg
1081 gaatttatgt gtgtgaagga gttaatttga ttgggaaaaa cagaaaagag gtggaattaa
1141 ttgttcaaga gaaaccattt actgttgaga tctcccctgg accccggatt gctgctcaga
1201 ttggagactc agtcatgttg acatgtagtg tcatgggctg tgaatcccca tctttctcct
1261 ggagaaccca gatagacagc cctctgagcg ggaaggtgag gagtgagggg accaattcca
1321 cgctgaccct gagccctgtg agttttgaga acgaacactc ttatctgtgc acagtgactt
1381 gtggacataa gaaactggaa aagggaatcc aggtggagct ctactcattc cctagagatc
1441 cagaaatcga gatgagtggt ggcctcgtga atgggagctc tgtcactgta agctgcaagg
1501 ttcctagcgt gtaccccctt gaccggctgg agattgaatt acttaagggg gagactattc
1561 tggagaatat agagtttttg gaggatacgg atatgaaatc tctagagaac aaaagtttgg
1621 aaatgacctt catccctacc attgaagata ctggaaaagc tcttgtttgt caggctaagt
1681 tacatattga tgacatggaa ttcgaaccca aacaaaggca gagtacgcaa acactttatg
1741 tcaatgttgc ccccagagat acaaccgtct tggtcagccc ttcctccatc ctggaggaag
1801 gcagttctgt gaatatgaca tgcttgagcc agggctttcc tgctccgaaa atcctgtgga
1861 gcaggcagct ccctaacggg gagctacagc ctctttctga gaatgcaact ctcaccttaa
1921 tttctacaaa aatggaagat tctggggttt atttatgtga aggaattaac caggctggaa
1981 gaagcagaaa ggaagtggaa ttaattatcc aagttactcc aaaagacata aaacttacag
2041 cttttccttc tgagagtgtc aaagaaggag acactgtcat catctcttgt acatgtggaa
2101 atgttccaga aacatggata atcctgaaga aaaaagcgga gacaggagac acagtactaa
2161 aatctataga tggcgcctat accatccgaa aggcccagtt gaaggatgcg ggagtatatg
2221 aatgtgaatc taaaaacaaa gttggctcac aattaagaag tttaacactt gatgttcaag
2281 gaagagaaaa caacaaagac tatttttctc ctgagcttct cgtgctctat tttgcatcct
2341 ccttaataat acctgccatt ggaatgataa tttactttgc aagaaaagcc aacatgaagg
2401 ggtcatatag tcttgtagaa gcacagaagt caaaagtgta gctaatgctt gatatgttca
2461 actggagaca ctatttatct gtgcaaatcc ttgatactgc tcatcattcc ttgagaaaaa
2521 caatgagctg agaggcagac ttccctgaat gtattgaact tggaaagaaa tgcccatcta
2581 tgtcccttgc tgtgagcaag aagtcaaagt aaaacttgct gcctgaagaa cagtaactgc
2641 catcaagatg agagaactgg aggagttcct tgatctgtat atacaataac ataatttgta
2701 catatgtaaa ataaaattat gccatagcaa gattgcttaa aatagcaaca ctctatattt
2761 agattgttaa aataactagt gttgcttgga ctattataat ttaatgcatg ttaggaaaat
2821 ttcacattaa tatttgctga cagctgacct ttgtcatctt tcttctattt tattcccttt
2881 cacaaaattt tattcctata tagtttattg acaataattt caggttttgt aaagatgccg
2941 ggttttatat ttttatagac aaataataag caaagggagc actgggttga ctttcaggta
3001 ctaaatacct caacctatgg tataatggtt gactgggttt ctctgtatag tactggcatg
3061 gtacggagat gtttcacgaa gtttgttcat cagactcctg tgcaactttc ccaatgtggc
3121 ctaaaaatgc aacttctttt tattttcttt tgtaaatgtt taggtttttt tgtatagtaa
3181 agtgataatt tctggaatta gaaaaaaaaa aaaaaaaaaa
vascular cell adhesion protein 1 isoform a precursor [Homo sapiens]
NP_001069.1 GI:4507875
(SEQ ID No: 10)
      1 mpgkmvvilg asnilwimfa asqafkiett pesrylaqig dsvsltcstt gcespffswr
    61 tqidspingk vtnegttstl tmnpvsfgne hsylctatce srklekgiqv eiysfpkdpe
  121 ihlsgpleag kpitvkcsva dvypfdrlei dllkgdhlmk sqefledadr ksletkslev
  181 tftpviedig kvlvcraklh idemdsvptv rqavkelqvy ispkntvisv npstklqegg
  241 svtmtcsseg lpapeifwsk kldngn1qh1 sgnatltlia mrmedsgiyv cegvnligkn
  301 rkevelivqe kpftveispg priaaqigds vmltcsvmgc espsfswrtq idsplsgkvr
  361 segtnstltl spvsfenehs ylctvtcghk klekgiqvel ysfprdpeie msgglvngss
  421 vtvsckvpsv ypldrleiel lkgetileni efledtdmks lenkslemtf iptiedtgka
  481 lvcqaklhid dmefepkqrq stqtlyvnva prdttvlvsp ssileegssv nmtclsqgfp
  541 apkilwsrql pngelqplse natltlistk medsgvylce ginqagrsrk eveliiqvtp
  601 kdikltafps esvkegdtvi isctcgnvpe twiilkkkae tgdtvlksid gaytirkaql
  661 kdagvyeces knkvgsqlrs ltldvqgren nkdyfspell vlyfasslii paigmiiyfa
  721 rkanmkgsys lveaqkskv
```

In various embodiments, the present methods and protein analysis may be carried out with or on a system incorporating computer and/or software elements configured for performing logic operations and calculations, input/output operations, machine communications, statistical analysis, detection of gene or protein expression levels and analysis of the measured levels and/or the like. Such system may also be used to generate a report, determinations of the total expression levels measured, the comparison with any reference levels, and calculation of the median levels of gene and gene product expression levels. It will be appreciated by one of skill in the art that various modifications are anticipated by the present embodiments.

In various embodiments, the methods described carried out on a computer readable storage medium having computer readable program code embodied in the medium to carry out the methods and determinations of protein concentration and/or patient dosage classification.

In some embodiments, protein concentration is measured by ELISA by optical density (O.D.) or fluorescent intensity. In some embodiments, the clinician drops a plasma serum or whole blood sample on a reader such as a Radbiochip described in Kim, D., Marchetti, F., Chen, Z., Zaric, S., Wilson, R. J., Hall, D. A., Gaster, R. S., Lee, J. R., Wang, J., Osterfeld, S. J., Yu, H., White, R. M., Blakely, W. F., Peterson, L. E., Bhatnagar, S., Mannion, B., Tseng, S., Roth, K., Coleman, M. A., Snijders, A. M., Wyrobek, A. J., Wang, S. X. Nanosensor dosimetry of mouse blood proteins after exposure to ionizing radiation. Scientific Reports(Nature). 3:2234, 2013, and the reader would determine OD or fluorescent intensity, followed by software which would run Ensemble vote predictions for which dose class a person should be classified. RF analysis is employed to determine the relative discrimination informativeness of each marker, but could be used for dose category prediction. In some embodiments, for dose prediction the ensemble majority vote from supervised classifiers is used.

In various embodiments, a patient's predicted dose category is determined after a known exposure event, for which time since exposure would always be known. However, rrediction for time since exposure using methods herein are contemplated. Time prediction from markers may further involve regression models, or possibly classification models withhold as inputs.

In various embodiments, a computer-implemented software component carries out the methods described herein. In some embodiments, such method uses optical density (O.D.) or fluorescent intensity, dose prediction is made and the dose category is determined The software would be trained using blood sample data similar to the mouse data described herein, data from non-human primates, data from irradiated blood samples, or data from patients that have received known irradiation exposure (e.g., cancer, radiotherapy patients).

In one embodiment, the 1-4 protein signature panel may also be added to a larger biomarker panel comprising the detection of the genes or gene products. A method is described for identifying a patient with higher predicted probability of disease free survival. Methods for determining such disease-free survival may comprise: (a) measuring the amplification or expression level of each gene in the biomarker panel in a sample from a patient; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby a below-median expression level indicates a patient that has a higher predicted probability of disease free survival.

In one embodiment, a kit comprising probes for detection of expression levels of the 1-4 protein signature panel, wherein said probes provide for assessment of a subject's radiation exposure.

In other embodiments, a sample is obtained from a patient; an ELISA is conducted; FLT3LG, SAA1, CC3, and VCAM-1 protein concentrations in the patient blood sample are determined, for example, by using an optical density spectrophotometer reader and protein concentration is calculated e.g., ng/mL concentration using analysis software; the protein concentrations are next log transformed, mean-zero standardized, and then transformed through the classification algorithms and methods described herein in the Examples to produce prediction scores for the dose classification of the patient sample. The resulting prediction scores are normally distributed and equilibrated, and a prediction/probability is made for each possible dose class membership, with the greatest probability where the patient is classified. In some embodiments, the classification methods employ Ensemble methods which use 8 supervised votes, and the majority class determines the dosage classification. In other embodiments, it may be better to use Ensemble majority and weighted majority vote methods.

In various embodiments, following the determination of radiation dosage classification of the patient sample, the patient is then triaged according to the severity of the dosage received and then appropriate treatments are recommended and prescribed.

### EXAMPLE 1

### ELISA Biomarker Predictor Panel

It is generally believed that the best accuracy in assigning an individual with unknown exposure to ionizing radiation into the correct dose category for proper medical care requires panels of multiple radiation-responsive biomarkers. However, major uncertainties remain regarding the number of biomarkers required and the selection of the best methods for unsupervised dose class discovery and supervised dose class prediction.

This investigation was undertaken to evaluate classification analysis of gold standard ELISA proteomic data for four candidate markers considered for use on a nano-biochip for individual radiation dose prediction [2]. We selected the following four biomarkers for analysis by ELISA: 1) FLT3 ligand (Fms-related tyrosine kinase 3 ligand; FLT3LG), which is a hematopoietic growth factor and is used as a clinical indicator for bone marrow status; 2) Serum amyloid A (SAA1), which is a major acute phase protein that is expressed and regulated in response to tissue injury and inflammation; 3) CC3, also known as HTATIP2 (HIV-1 Tat interactive protein 2), which is an oxidoreductase with proapoptotic as well as antiangiogenic properties; and 4) VCAM-1 (vascular cell adhesion molecule 1), which plays a role in cell-cell recognition [3]. Random forests were used for calculating permutation-based importance scores, feature selection frequency counts during decision tree learning, and for generating unsupervised and supervised cluster representations of samples via eigenanalysis of decision tree-based proximity matrices. We also employed several feature filtering and selection techniques, and a variety of supervised classification methods for class prediction of irradiation dose category.

C57BL/6 inbred mice were exposed to 0 Gy, 1 Gy, 2 Gy, 3 Gy,or 6 Gy and peripheral blood plasma was obtain on day 1 (n=50) and day 5 after exposure (n=50) for evaluation by ELISA for 4 proteomic biomarkers (FLT3LG, SAA1, CC3, and VCAM) in triplicate with equal numbers of males and females in each dose-time group. Random forests (RF) were used for unsupervised analyses to evaluate biomarker informativeness, while 8 supervised classification techniques were compared for multiclass analyses of this 5-class problem (0,1,2,3,6 Gy). Classifiers included k nearestneighbor (kNN), naive Bayes classifier (NBC), linear discriminant analysis (LDA), learning vector quantization (LVQ1), least squares support vector machines (SVMLS), artificial neural networks (ANN), constricted particle swarm optimization (CPSO), and polytomous logistic regression (PLOG). Results indicate that gender and time since exposure were much less informative than the biomarker responses were for dose category assignments. For day 1, SAA1 and FLT3LG are almost equally informative when considering RF importance scores and supervised classification results. For day 5, FLT3LG dominated the importance scores, but VCAM, CC3 and SAA1 were, nevertheless, selected quite frequently during first and all node splits during RF decision tree generation. During feature filtration and selection, only FLT3LG was selected for the day 5 classification runs. Feature selection approaches using various inferential hypothesis testing approaches did not result in markedly different classification performance. For day 1 supervised classification analyses, the overall accuracies for each method were (in decreasing order): EMV (80%), PLOG (78%), LDA (77%), 5NN (76%), CPSO (76%), EWMV (75%), LVQ1 (72%), ANN (71%), NBC (65%), and SVMLS (49%). For day 5 supervised classification results, the accuracies were: LDA (100%), 5NN (100%), LVQ1 (100%), CPSO (100%), PLOG (98%), EMV (96%), EWMV (87%), ANN (75%), SVMLS (71%), and NBC (53%). These analyses demonstrate that RF bootstrapping to generate alternative realizations of training data and simultaneous random selection of features during node splitting in decision tree learning is a superior approach to unsupervised and supervised classification analysis, especially for evaluating biomarker dose informativeness. Our findings lay the groundwork as additional radiation biomarkers become available to improve the cluster structure of the data and to improve supervised classification performance.

### Methods

Animals and Treatments. C57BL/6 mice, 8 to 10 week old males and females, were purchased from Harlan Laboratories. Mice were housed under conventional conditions in microisolator filter-top cages. Animal rooms were provided with 10-12 air changes h-1 of 100% fresh conditioned air and maintained at 22° C.±1° C. with a relative humidity of 50%±20. Animals remained on 12:12-h full spectrum light:dark cycles and provided food (Lab Diet 5008 Mouse Chow) and reverse osmosis filtered water ad libitum. Mice were acclimated for a minimum of 2 weeks before sham treatment or exposure to ionizing radiation. The use of animals in the study was approved by the Institutional Animal Care and Use Committee (IACUC) of Lawrence Berkeley National Laboratory, which approved the protocols.

Mouse Irradiations. Mice were total-body-irradiated (TBI) using a Pantak 320 kV X-ray machine set at 300 kV and 10 mA. Mean weights before irradiation were 28.2 g±3.3 and 22.4 g±3.0 for males and females, respectively. Irradiations of mice were carried out in well-ventilated clear plastic rodent restrainers each containing a single mouse. Restrainers were placed on a turning table and mice were irradiated one or two at a time. 1, 2 and 3 Gy irradiations were carried out at a dose rate of 775 mGy/min and the turning table was set to 85 cm. 6 Gy irradiations were carried out at a dose rate of 1.9 Gy/min and the turning table was set to 60 cm. Sham-irradiated animals were treated in the same manner but not exposed to the radiation source. Dosimetry was performed using an Accu-Pro™ dosimeter.

Euthanasia and blood collection. At 24 h or 5 d post irradiation, mice were weighed (mean weights were 27.4 g±3.1 and 21.9 g±2.6 for males and females, respectively) and euthanized by CO2 asphyxiation followed by open thoracotomy. Blood was collected via intracardiac puncture with a heparin rinsed syringe. The average volume of blood collected was 738 μl and 660 μl for males and females, respectively. Tubes with collected peripheral blood were centrifuged at 400 g for 5 min, and plasma was collected, aliquoted, and preserved at −80° C. until use. The average volume of plasma collected was 321 μl and 288 μl for males and females, respectively.

Groups of 10 C57BL/6 mice (5 male and 5 female) underwent whole-body irradiation using X-rays (0.7 Gy/min dose rate) to doses of 0 (sham), 1, 2, 3, and 6 Gy (total=100 mice) with approval of the LBNL Animal Use Committee. Cardiac blood was collected at24 hours and 5 days after exposure and plasma prepared for protein ELISA analyses. Triplicate measures of plasma-based ELISA concentration for four protein biomarkers (FLT3LG, SAA1, CC3, VCAM-1) were obtained from each mouse at 24 h post-exposure (n=50) and 5 days post-exposure (n=50).

Protein bioassays. Total protein concentrations of the samples were measured via the bicinchoninic acid (BCA) method (Pierce). Radiation responses of blood protein biomarkers were measured using ELISA. All quality control concentrations were within 2SD of mean on all plates that were run.

FLT-3LG. Sandwich ELISA for mouse FLT-3LG was run according to manufacturer's instructions using a commercially available kit (R&D Quantikine Mouse*Flt-3 Ligand Immunoassay, cat #MFK00, Minneapolis, Minn., USA). The quality control provided with the kit was resuspended in distilled water, aliquoted, and frozen. An aliquot of the quality control was run in triplicates on each plate. The quality control gave an average concentration of −237 pg/mL.

SAA1. Sandwich ELISA for mouse SAA1 was run according to manufacturer's instructions using a commercially available kit (ALPCO Immunoassays, cat #41-SAAMS-E01, Salem, N.H., USA). The quality control provided with the kit was diluted 1:2000, aliquoted, and frozen. An aliquot of the quality control was run in triplicates on each plate. The quality control gave an average concentration of −215 μg/mL.

CC3. Sandwich ELISA for mouse CC3 was run according to manufacturer's instructions using a commercially available kit (Alpha Diagnostic International Mouse C3, cat #6270, San Antonio, Tex. USA). The quality control provided with kit (mouse serum) was pooled from 6 ELISA kits, aliquoted, and placed at 4° C. An aliquot of the quality control was run in triplicates on each plate. The quality control gave an average concentration of ˜66 ng/mL.

VCAM-1. Sandwich ELISA for mouse VCAM-1 was run according to manufacturer's instructions using a commercially available kit (Abnova, cat #KA0428, Taipei City, Tiwan). A quality control was not provided with the kit. A quality control was made by using 10 μl of plasma collected from a sham irradiated mouse from a pilot study. The plasma was diluted 1:400 with VCAM sample diluent buffer, aliquoted, and frozen. An aliquot of the quality control was run in triplicates on each plate. The quality control gave an average concentration of −800 μg/mL

Data Collection. ELISA plates were read using TECAN Infinite M200 plate reader using the TECAN Magellan software. Data obtained from Magellan was exported into Microsoft Excel.

Biomarker Transformations. Triplicate ELISA concentration measurements for each mouse were collapsed into an average value. Within each biomarker, the continuously-scaled averages were log-transformed and then mean-zero standardized using the mean and standard deviation over all mice. For notation purposes, the log-transformed variant of FLT3LG was lnflt31g, and the mean-zero standardized value was then termed zlnflt31g. Therefore, the final variable names of the four log-transformed mean-zero standardized biomarker ELISA concentrations were zlnflt31g, zlnsaal, zlncc3, zlnvcam, which were used in all analyses.

Statistical Analyses. We used the linear discriminant analysis (LDA) and k-nearest neighbors (k=7, i.e., “7NN”) modules of Stata Version 12 (College Station, TX) for classification analysis. The MAUCROC algorithm for Stata was used for generating receiver operator characteristic curve (ROC) area under the curve (AUC). Classification and AUC runs were made for all possible pairs of dose classes (e.g., 0 vs. 1, 0 vs. 2, . . . ,3 vs. 6) as well as all one-against-remaining class comparisons (e.g., 0 vs. other, 1 vs. other, . . . , 6 vs. other) for the Day 1 and Day 5 data.

Unsupervised and Supervised Random Forest Analysis of Biomarker Informativeness. Random forests (RF) were used to generate importance scores and frequency of biomarker (feature) selection in first node splits and all node splits within the trees employed in a forest[4]. A total of 1,000 trees was used for each forest, and for each node split jtry=\sqrt{p}features were randomly selected and evaluated with the Gini index to identify the optimal cutpoint value for splitting. During tree generation, node splitting was performed until each daughter node had either one object or multiple objects with class purity. Supervised clustering results were based on eigenanalysis of the proximity matrix and presented in the form of 2D principal component score plots with varying symbols(colors) assigned to objects based on their true class labels. For unsupervised cluster analysis, the dataset being analyzed was augmented with n simulated objects by randomly selecting feature values from the observed n objects (within the same feature), such that the final dataset contained a total of 2n objects. Objects in the original dataset were assigned class 1 and objects in the augmented dataset were assigned to class 2. Eigenanalysis was then performed on the proximity matrix of the 2n objects, and 2D score plots weregenerated for the first n original objects in class 1 using a single symbol(color).

Biomarker Filtering from Class comparisons. Let xᵢ=(xᵢ₁, xᵢ₂, . . . , xᵢₚ) be an object (mouse) with p(j=1,2, . . . , p) features (biomarkers), n(i=1,2, . . . , n) the total number of objects (mice), and Ω(ω=1,2, . . . , Ω) be the total number of classes. In addition, let M=Ω(Ω−1)/2 be the possible pairs of class comparisons and M=Ω be all possible one-against-all remaining class comparisons (m=1,2, . . . , M). For each mth class comparison, the top N, biomarkers with the greatest informativeness were identified. Informativeness was based on the T-test, Mann-Whitney test, F-test, Kruskal-Wallis test, Gini index and entropy in the form of information gain[5]. For statistical tests, Nₘ was equal to the number of biomarkers for which pⱼ≦0.05. A list of non-redundant biomarkers among the N=N₁+N₂+ . . . +Nₘ+ . . . +N_{M} biomarkers was then constructed. (For large gene lists, we commonly identify 150 unique biomarkers from M sets of Nₘ=150/M biomarkers). Table 4 lists the number of biomarkers filtered for the M possible class comparisons. It warrants noting that the biomarkers from various class comparisons can be redundant, so a unique list is obtained from the M comparisons.

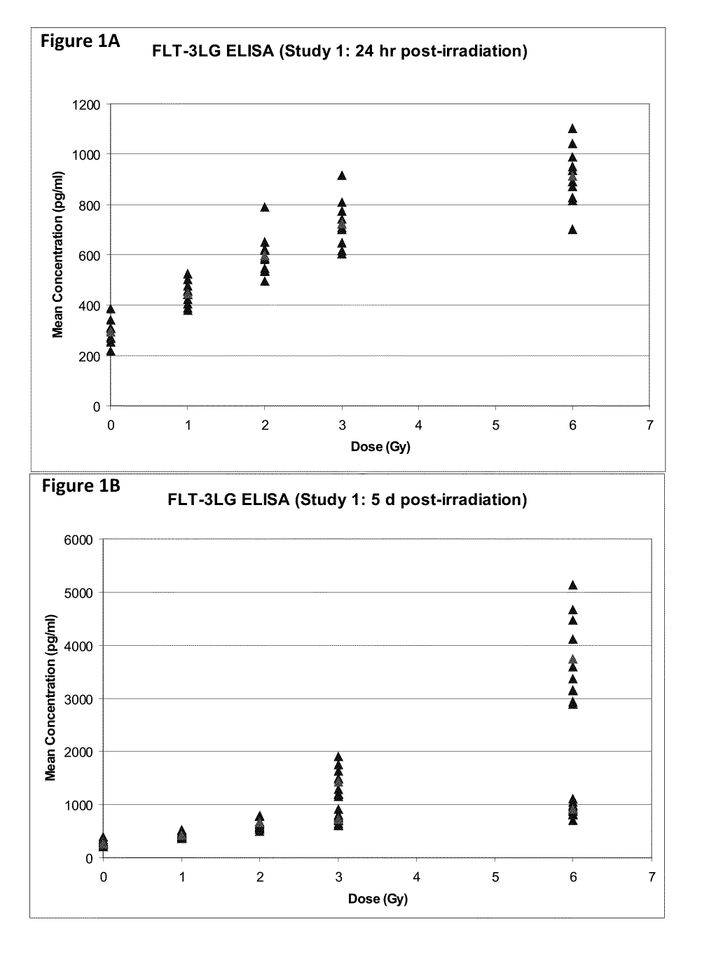

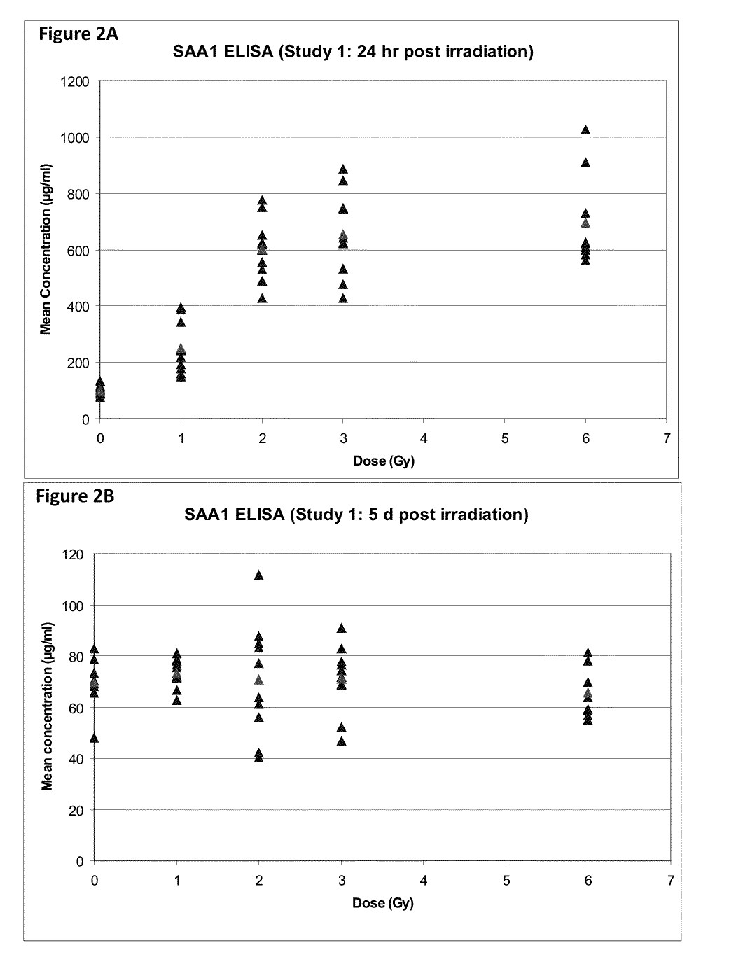

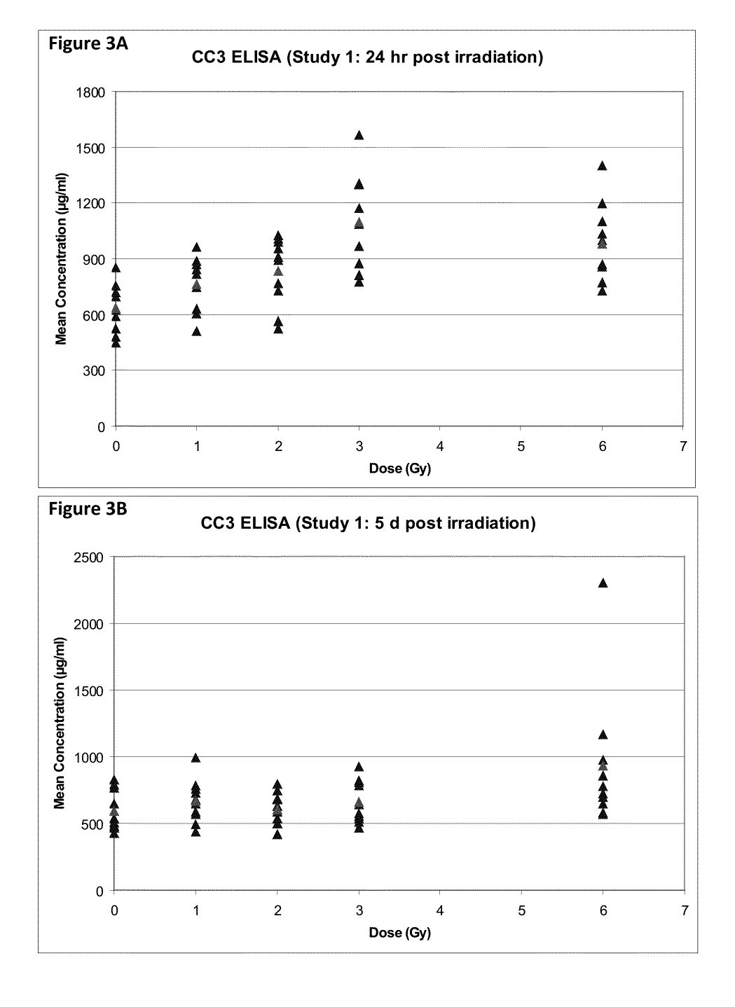

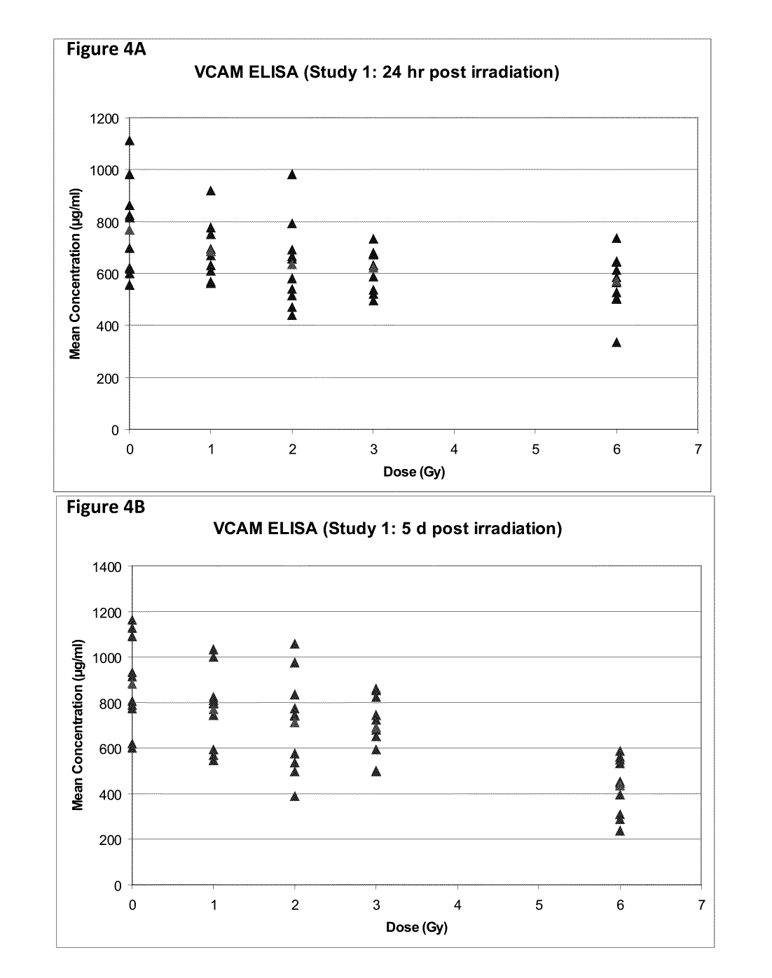

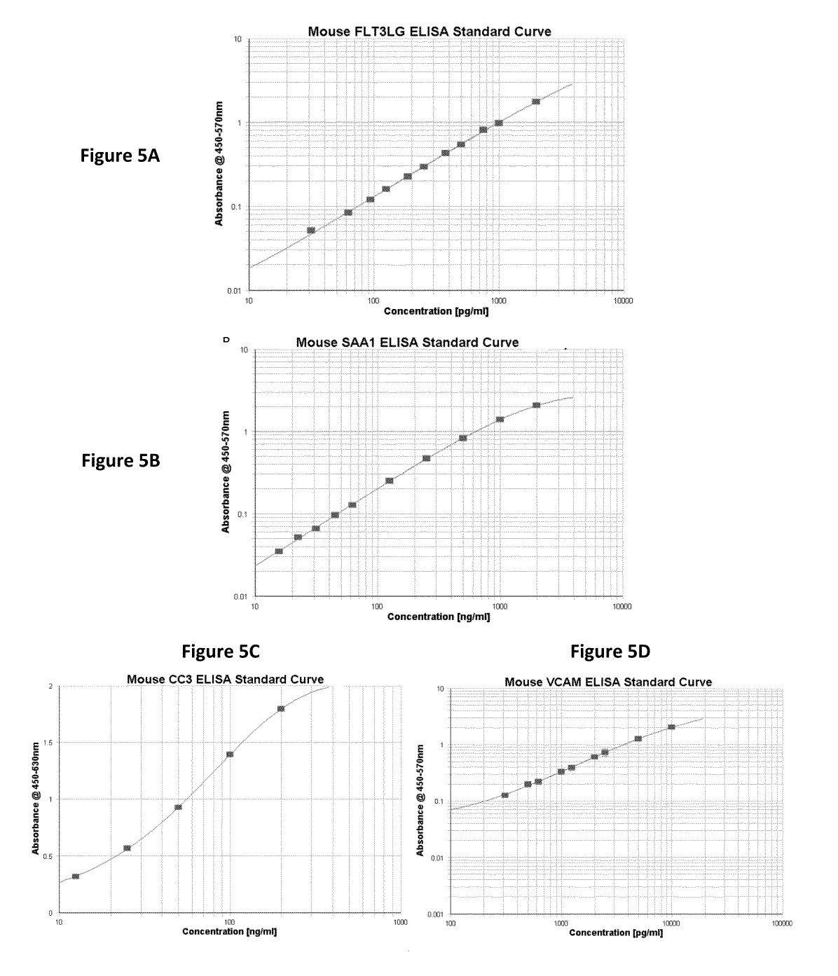

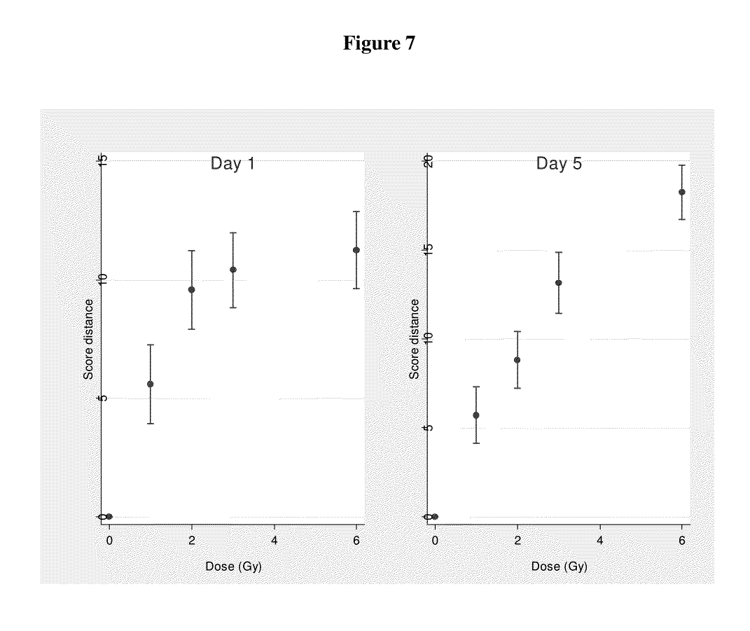

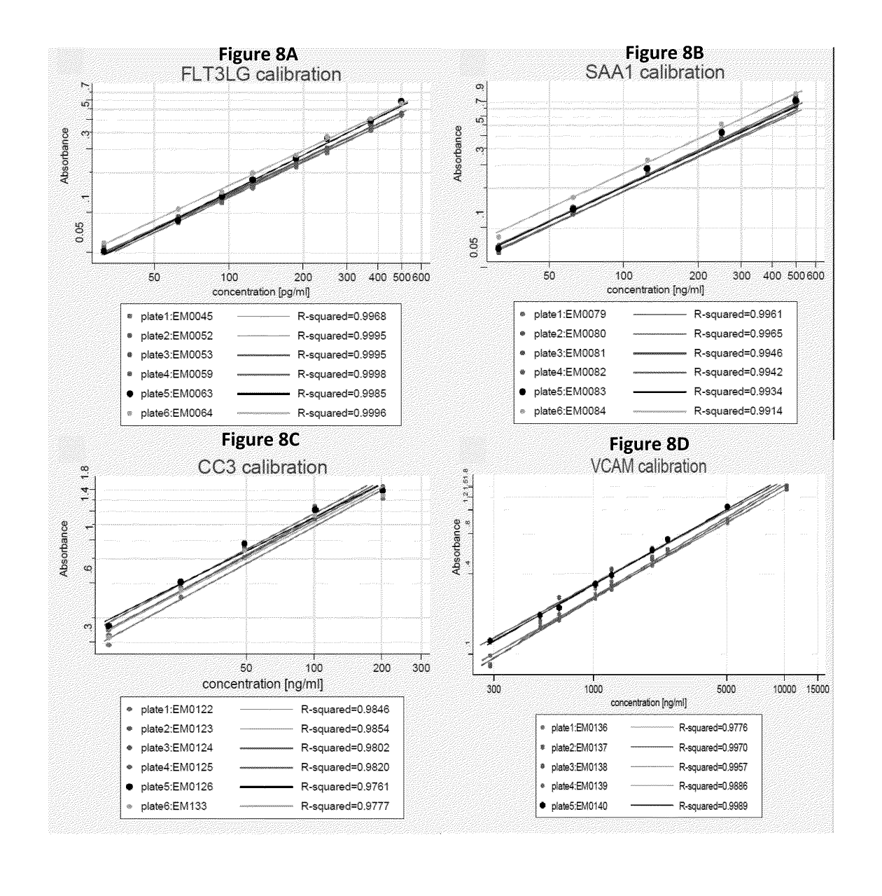

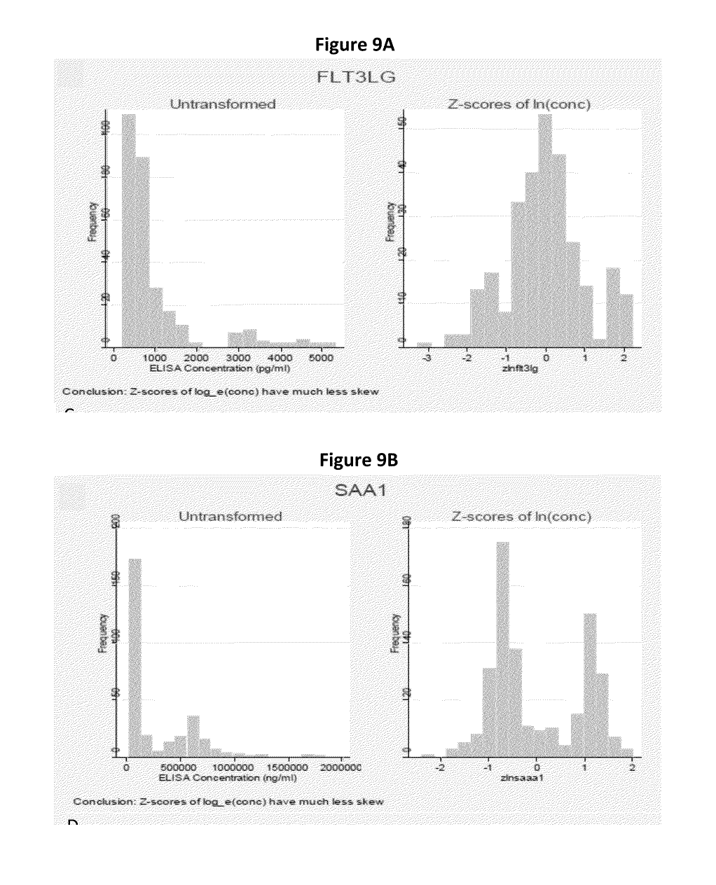

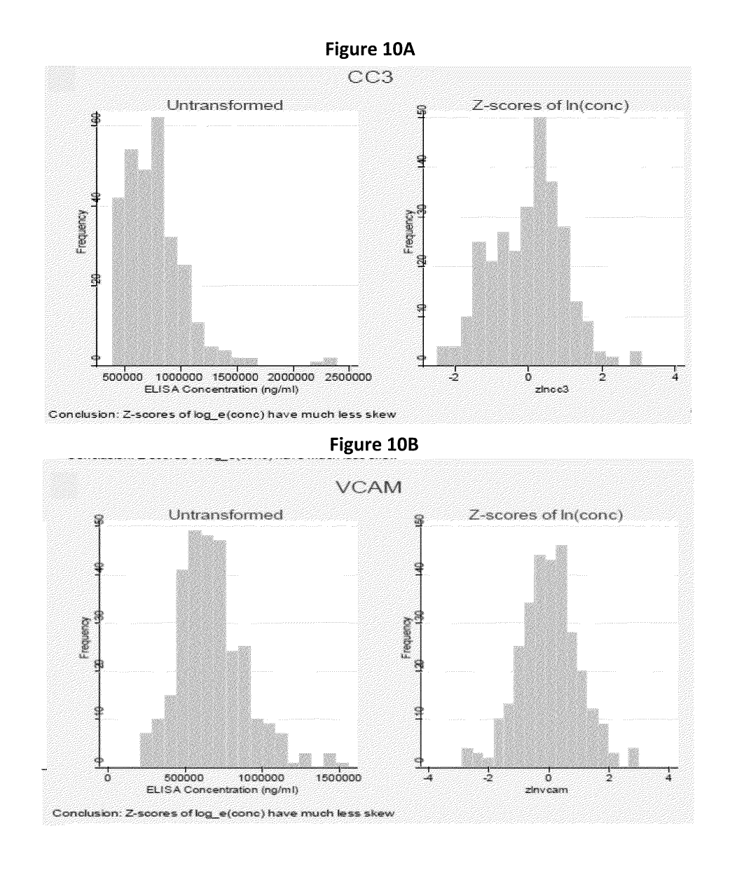

Results. Results for the selected protein biomarkers (FLT-3LG, SAA1, CC3 and VCAM) in mouse plasma represent 5 mice per group (dose and sampling time-point) derived from four independent experiments. Results shown for FLT-3LG (FIG. 1), SAA1 (FIG. 2), CC3 (FIG. 3), and VCAM (FIG. 4) demonstrate a progressive dose dependent response, either at 1 and/or 5 days post irradiation. The standard curves used to analyze ELISA results for each biomarker are shown in FIG. 5. Dose-dependent separation in canonical score values of irradiated animals from controls is shown in FIG. 7. Score distance in FIG. 7 represents a distance (in canonical score) between medians of distributions of irradiated and control animals.

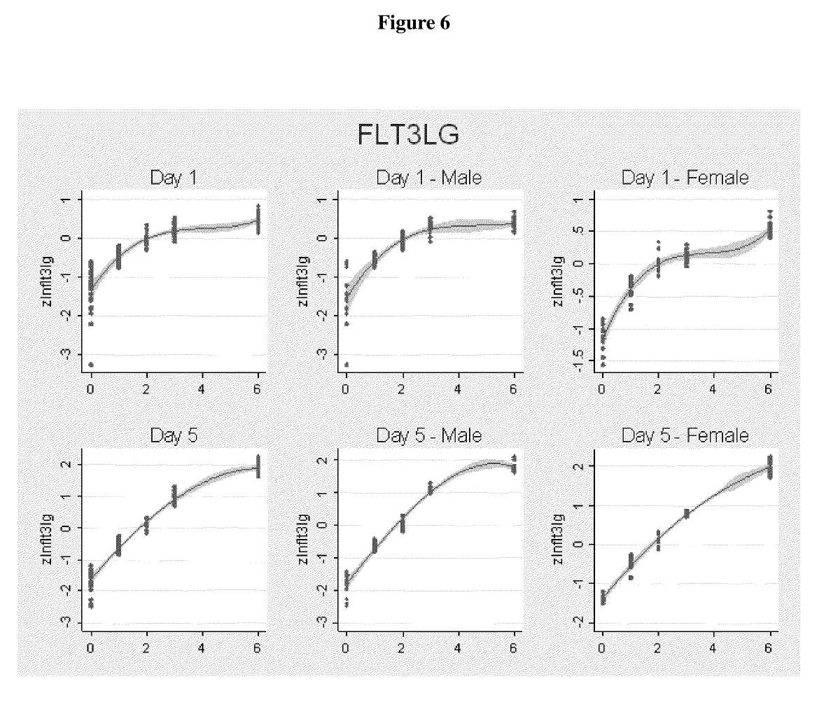

FLT-3LG. As shown in FIG. 1, ionizing radiation significantly increased plasma levels of FLT-3LG at all doses tested at both 24 hr and 5 days (p<0.0001 vs. sham; range, 9.1E-06 to 7.2E-10). At 24 hr, mean fold changes with respect to sham were: 1.5, 2.0, 2.5 and 3.1 for 1, 2, 3, and 6 Gy, respectively. An even stronger induction was seen at 5 days when mean fold changes with respect to sham were: 1.6, 2.6, 5.6 and 14.6 for 1, 2, 3, and 6 Gy, respectively. Dose response curves (FIG. 6) are shown for male and female mice on day 1 and day 5 after irradiation. 4A shows the dose response for FLT-3LG in terms of average concentration separated by dose, time point and gender. Concentrations for all doses at both 1 and 5 days after irradiation increased significantly with respect to sham animals in both males and females.

SAA1. As shown in FIG. 2, ionizing radiation significantly increased plasma levels of SAA1 at all doses tested at 24 hr after irradiation (p<0.0001 vs. sham; range, 1.2E-07 to 4.3E-8). At 24 hr, mean fold changes with respect to sham were: 2.5, 6.2, 6.6 and 7.0 for 1, 2, 3, and 6 Gy, respectively. At day 5, SAA1 levels in irradiated mice had returned to baseline levels and were not different from sham, irrespective of radiation dose. Table 4B shows the dose response for SAA1 in terms of average concentration separated by dose, time point and gender. Concentrations for all doses at 1 day after irradiation increased significantly with respect to sham animals in both males and females. Overall, there was no significant difference with respect to sham at 5 days post irradiation. However at 5 days post irradiation, 1 Gy males and 3 Gy females showed a significant difference in SAA1 concentration compared to sham.

CC3. As shown in FIG. 3, ionizing radiation significantly increased plasma levels of CC3 at all doses tested at 24 hr after irradiation (p<0.05 vs sham; range, 0.04 to 0.0003). At 24 hr, mean fold changes with respect to sham were: 1.1, 1.2, 1.6 and 1.5 for 1, 2, 3, and 6 Gy, respectively. At day 5, CC3 levels in irradiated mice appeared to have returned to baseline levels and were not different from sham, irrespective of the radiation dose. Table 4C shows the dose response for CC3 in terms of average concentration separated by dose, time point and gender. Concentrations for all doses at 1 day after irradiation increased significantly with respect to sham animals in both males and females. Overall, there was no significant difference in CC3 concentration with respect to sham at 5 days post irradiation. However, at 5 days post irradiation, 6 Gy females showed a significant difference in CC3 concentration compared to sham.

VCAM. As shown in FIG. 4, ionizing radiation induced a dose-related decrease of plasma levels of VCAM at both 24 hrs and 5 days. At 24 hrs, VCAM levels were significantly reduced with respect to sham for 3 (p<0.02) and 6 Gy (p<0.004). At 5 days, decreases were significant for 2 (p<0.05), 3 (p<0.02) and 6 Gy (P<0.0001). At 24 hrs, mean fold changes with respect to sham ranged from −1.1 to −1.3 across dose groups. Table 4D shows the dose response for VCAM-1 in terms of average concentration separated by dose, time point and gender. At 1 day post irradiation, VCAM levels decreased significantly in 3 and 6 Gy animals with respect to sham. Overall, at 5 days post irradiation, there was a significant decrease in 2, 3 and 6 Gy animals compared to sham. However, at 5 days post irradiation, there was not a significant decrease observed in 3 Gy irradiated males or in 2 Gy irradiated females with respect to sham. There was however, a significant decrease in VCAM concentration observed in 1 Gy females at 5 days post irradiation.

Table 5 lists receiver operator characteristic (ROC) area under the curve (AUC) for all possible 2-class comparisons of triplicate ELISA Day 1 response using linear discriminant analysis (LDA) and k-nearest neighbor (k=7, i.e. “7NN”) with leave-one-out cross validation (LOOCV) for Day 1 and Day 5. For Day 1 data, mean univariate AUCs for FLT3LG and SAA1 exceeded 90%, while mean univariate AUC for CC3 and VCAM were less than 90%. Average multivariate AUC for both classification methods (LDA and 7NN) exceeded 95%. For Day 5 data, mean univariate AUCs for FLT3LG exceeded 90%, while mean univariate AUC for SAA1, CC3, and VCAM were less than 90%. Average multivariate AUC for both classification methods (LDA and 7NN) was equal to 100%. Table Mists receiver operator characteristic (ROC) area under the curve (AUC) for one-against-other classification analyses for triplicate ELISA Day 1 and 5 response using k-nearest neighbor (k=7, i.e. “7NN”) and linear discriminant analysis (LDA) with leave-one-out cross validation (LOOCV). For Day 1 data, mean univariate AUC for FLT3LG was 93% for 7NN and was 79% for LDA. Mean univariate AUC for SAA1, CC3, and VCAM were less than 90% for both 7NN and LDA. Average multivariate AUC for 7NN and LDA was 95% and 85%, respectively. For Day 5 data, mean univariate AUC for FLT3LG was 100% for 7NN and was 81% for LDA. Mean univariate AUC for SAA1, CC3, and VCAM were less than 80% for both 7NN and LDA. Average multivariate AUC for 7NN and LDA was 100% and 80%, respectively.

For Day 1 data, we expected classifier breakdown for the 2 vs. 3 vs. 6 Gy dose comparisons, since the dose-response results do not reveal clear separation of response of FLT3LG and SAA1 at greater doses. On day 5, FLT3LG contributes to the majority of discrimination due to the greater separation between mean responses over the entire dose range. Overall, LDA results were less appealing than results from 7NN classification. The non-parametric k-nearest neighbor classifier has the ability to go within clusters of sample and correctly classify each test sample left of training during LOOCV using the majority true class label among k closest neighbors in Euclidean space. Whereas, LDA assumes that the number of samples used is large enough to ensure that the pooled covariance matrix approximates a multivariate normal distribution and that there are no outliers that can bias the results through leveraging.

FLT3LG. Our results showed that ionizing radiation significantly increased plasma levels of FLT-3LG at all doses tested at both 24 hr and 5 days. The strongest induction of FLT-3LG levels was observed at 5 days post irradiation. These results are in agreement with Prat et al.(2005) in BALB/c mice where it was demonstrated that FLT-3LG levels peaked at 3 and 7 days post irradiation. Our results were also in agreement with Sugimoto (2001) who showed that C3H/HeN mice irradiated with 15 Gy showed a significant increase in serum FLT-3LG levels with a peak at 6 days post irradiation.

SAA1. Our results showed that ionizing radiation significantly increased plasma levels of SAA1 at all doses tested at 24 hr after irradiation. At day 5, SAA1 levels in irradiated mice had returned to baseline levels and were not different from sham, irrespective of radiation dose. These results are in agreement with findings of Ossetrova and Blakely (2009) and Ossetrova et al (2010) in BALB/c mice.

CC3. Our results showed that ionizing radiation significantly increased plasma levels of CC3 at all doses tested at 24 hr after irradiation. At day 5, CC3 levels in irradiated mice appeared to have returned to baseline levels and were not different from sham, irrespective of the radiation dose. Rithidech et al (2009) had used 2-D gel to identify a significant increase in plasma CC3 levels at 7 days after exposure to 3 Gy in CBA/CaJ mice, suggesting possible genetic variation in time response.

VCAM-1. Our results showed that ionizing radiation induced a dose-related decrease of plasma levels of VCAM at both 24 hrs and 5 days. Rithidech et al (2009) used 2-D gel to identify a significant increase in plasma VCAM at 7 days after exposure to 3 Gy in CBA/CaJ, suggesting that biomarker response may show genetic variation.

### EXAMPLE 2

### Determining Informativeness of Biomarker Predictor Panel

RF analysis allowed us to evaluate the informativeness of biomarker response at 1 and 5 days after exposure for the purpose of dis-criminating the 5 classes of dose. The advantage of RF lies in the strength of bootstrapping multiple realizations of the data and each time randomly selecting features for Gini evaluation for each node splitting in order to generate thousands of decision trees in a forest. RF's do not overfit data and typically are the most reliable approach for generalizing results to future unobserved test data, which is mostly due to their conservativeness hinged to bootstrapping and random feature selection-evaluation during tree generation. Importance scores are permutation-based and offer a means of evaluating feature informativeness based on null and alternative distributions, while the frequency of feature selection during first node splits and all node splits provides additional information regarding feature informativeness—because if a feature results in strong class separation, it will likely be identified via Gini in the first node split and numerous other splits within a decision tree. The supervised and unsupervised clustering results presented in 2D score plots of the proximity matrix reveal the cluster structure objects based on training data with intact class labels and simulated class labels. Table 1 shows the number of class comparisons, M, and number of biomarkers, Nₘ, filtered for each comparison.

Selection of Filtered Biomarkers for Supervised Classification. A stepwise greedy plus-take-away (“Greedy PTA”) method using a plus 1 take away 1 heuristic[6] was used for selecting biomarkers from the unique list of filtered biomarkers described above. (This step requires biomarkers to be mean-zero standardized over the n objects). Forward stepping was carried out to add(delete) the most(least) important biomarkers for class separability based on squared Mahalanobis distance and the F-to-enter and F-remove statistics. Biomarkers were entered into the model if their standardized expression resulted in the greatest Mahalanobis distance between the two closest classes, and their F-to-enter statistic exceeded F=3.84. At any step, a biomarker was removed if its F-to-enter statistic was less than the F-to-remove criterion of F=2.71. When done, there were N unique biomarkers which were jointly statistically significantly different between all classes, which also happened to provide the greatest multivariate Mahalanobis distance-based class separation. We also selected the “Best ranked N” biomarkers for supervised classification runs, where N was set equal to the number of unique biomarkers selected during greedy PTA.

Supervised Classification Analysis. Eight supervised classification techniques were employed for multiclass analysis of a 5-class problem (0 Gy, 1 Gy, 2 Gy, 3 Gy, and 6 Gy). These included k nearest neighbor (kNN), naïve Bayes classifier (NBC), linear discriminant analysis (LDA), learning vector quantization (LVQ1), least squares support vector machines (SVMLS), artificial neural networks (ANN), constricted particle swarm optimization (CPSO), and polytomous logistic regression (PLOG)[7,8,9,10,11,12,13,14]. For kNN, k was set equal to 5 (“5NN”), and for the LVQ1 we used a single prototype per class. For SVMs, we used an L2 soft norm least squares approach. A weighted exponentiated RBF kernel was employed to map samples in the original space into the dot-product space, given as

$$K ( x , {x}^{T} ) = exp ( - \frac{γ}{m} ‖ x - {x}^{T} ‖ ) ,$$

where m=#features. Such kernels are likely to yield the greatest class prediction accuracy providing that a suitable choice of γ is used. To determine an optimum value of γ for use with RBF kernels, a grid search was done using incremental values of γ from 2⁻¹⁵, 2⁻¹³, . . . , 2³ in order to evaluate accuracy for all training samples. We also used a grid search in the range of 10⁻², 10⁻¹, . . . , 10⁴ for the SVM margin parameter C. The optimal choice of C was based on the grid search for which classification accuracy was the greatest, resulting in the optimal value for the separating hyperplane and minimum norm ∥ξ∥ of the slack variable vector. SVM tuning was performed by taking the median of parameters during grid search iterations when the test sample misclassification rate was zero. For the ANN classifier, the logistic activation function was used with each hidden node, and the softmax function used to compute class membership probabilities for output node weight connections. We also used 500 sweeps with a grid search for each ANN model in which the learning rate E and momentum a ranged from 2⁻⁹, 2⁻⁸, . . . , 2⁻¹. The grid search for ANNs also included an evaluation of error for a variable number of hidden nodes in the single hidden layer, which ranged from the number of training features (i.e., the length of input vector for each sample) down to the number of output nodes, incremented by −2. In cases when there were multiple valuesof grid search parameters for the same error rate, we used the median value. Leave-one-out cross validation was employed for all runs.

Ensemble Techniques for Supervised Classifier Fusion. Classifiers were trained with the same feature sets, and then classifier votes were combined using the ensemble majority voting (EMV) and ensemble weighted majority voting (EWMV) ensemble combination techniques[15]. Let d_{l,ω}(x)in{0,1} be the decision rule for an object by the lth classifier (l=1,2, . . . , L) for class ω. The support for EMV and EWMV, respectively, is functionally composed as

$$\begin{matrix}{μ}_{ω} ( x ) = {\sum }_{l = 1}^{L} {d}_{l , ω} ( x ) , {d}_{l , ω} ( x ) ∈ \{ 0 , 1 \} , & ( 1 ) \\ {μ}_{ω} ( x ) = {\sum }_{l = 1}^{L} {w}_{l} {d}_{l , ω} ( x ) , {d}_{l , ω} ( x ) ∈ \{ 0 , 1 \} , & ( 2 )\end{matrix}$$

where wₗ is the normalized weight reflecting the accuracy of the lth classifier. Here, accuracy is based on the proportion of classified test objects assigned to the diagonal of the confusion matrix divided by the number of test objects. Let the ensemble decision for object x be E(x [symbol] ω). The decision rule for test object x is

E(x [symbol] ω)≡ω ∈ Ω arg max{μ_{ω}(x)}. (3)

Results of ensemble methods are presented under the classifier names EMV and EWMV in tabular form with results of the individual classifiers.

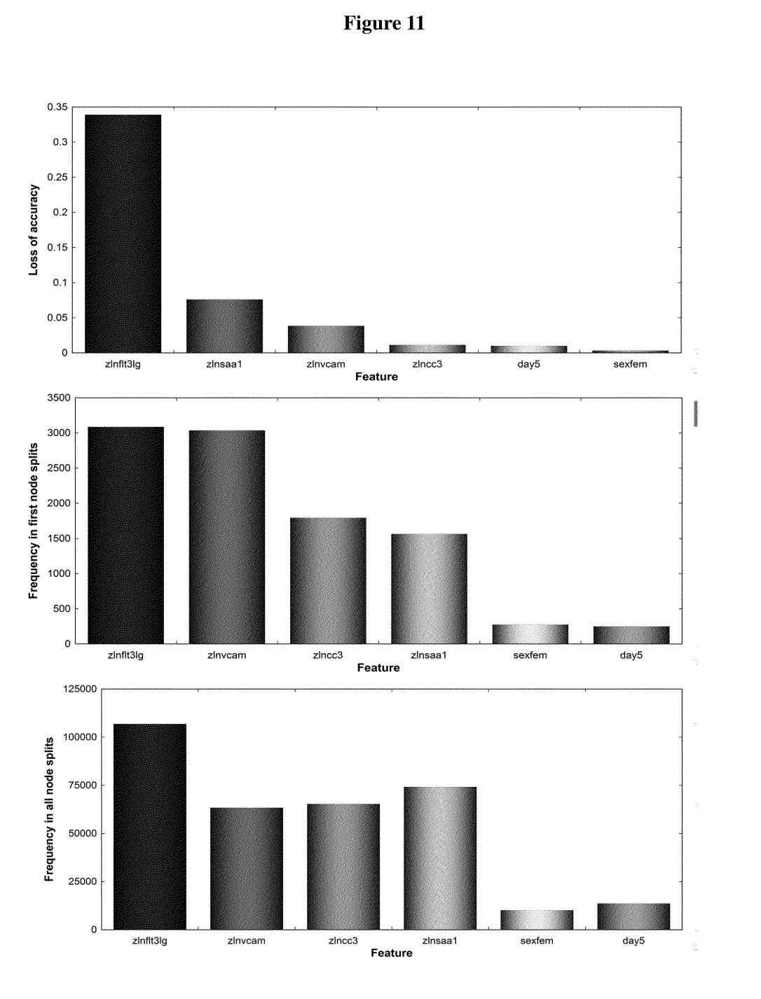

FIG. 11 illustrates RF importance scores (top panel) and frequency of feature selection during first node splits and all nose splits within 10,000 trees that were generated. The top panel in FIG. 11 shows RF importance scores for binary variables representing female gender (sexfem, 0-male, 1-females), day5 (0-day 1, 1-day 5), and continuously scaled ELISA concentrations of the biomarkers FLT3LG, SAA1, CC3, and VCAM. Notice that for both times after exposure, the FLT3LG biomarker response was more informative than the other biomarkers. In addition, gender and time after exposure (1 day or 5 days) were less informative for classification when compared with the biomarkers. The middle and bottom panels of FIG. 11 illustrate the frequency of selection of each feature during first node splits (10,000 total) and all node splits. It was interesting to observe that VCAM was selected more frequently for first node splits when compared with the SAA1 biomarker. For all node splits, we observed that the four biomarkers were selected much more frequently than the binary variables for gender and time after exposure.

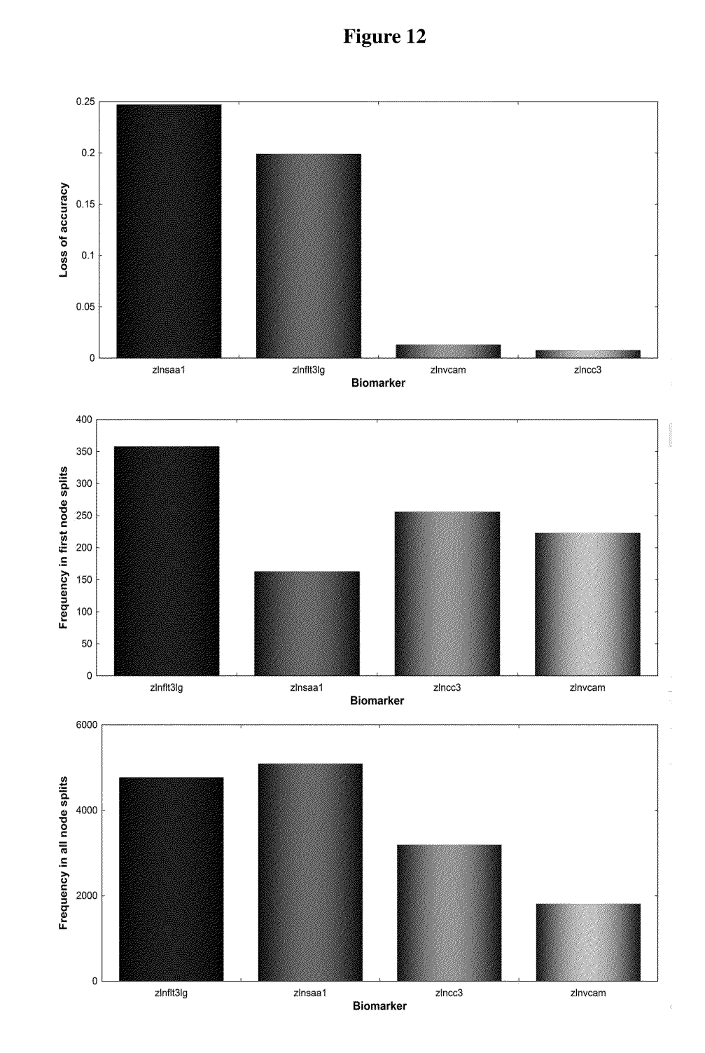

FIG. 12 shows the RF importance scores and frequency of biomarker selection during first node splits and all nodes splits for biomarker response at 1 day after exposure based on 1,000 trees. At day 1, SAA1 was the most informativebiomarker for the 5-class problem followed by FLT3LG. The VCAM and CC3 biomarkers were selected less frequency during node splitting, which is in agreement with the importance scores.

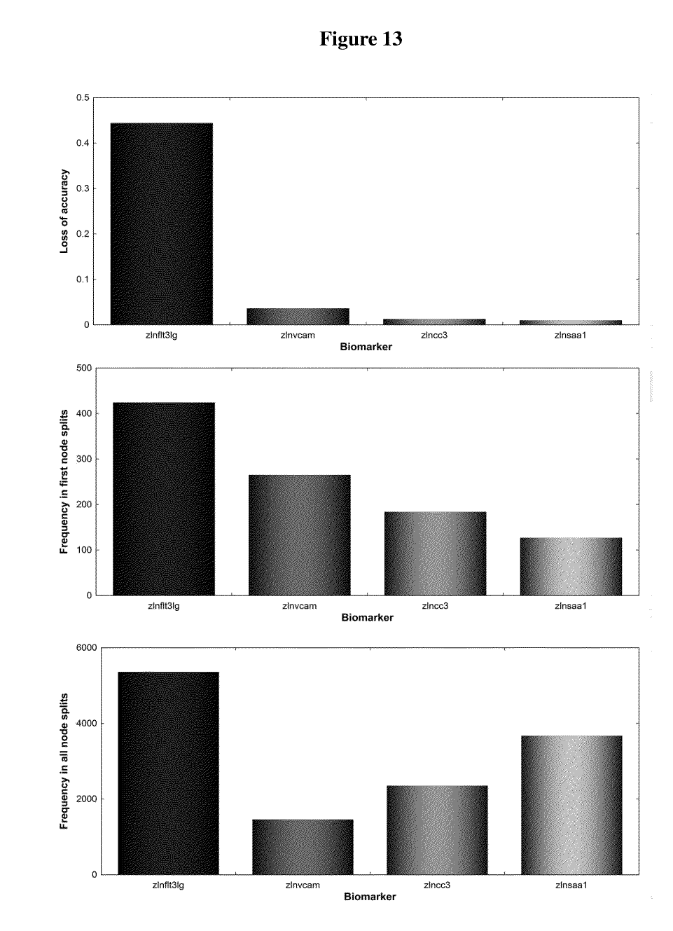

FIG. 13 shows the classification informativeness of biomarker response at 5 days after irradiation. The importance score for FLT3LG greatly exceeded importance for the other 3 biomarkers, and this was expected because of the strongly linear response of FLT3LG over the dose range 0-6 Gy. The VCAM and CC3 biomarker were selected more frequently than SAA1 during tree first node splits and less frequently than SAA1 during all node splits.

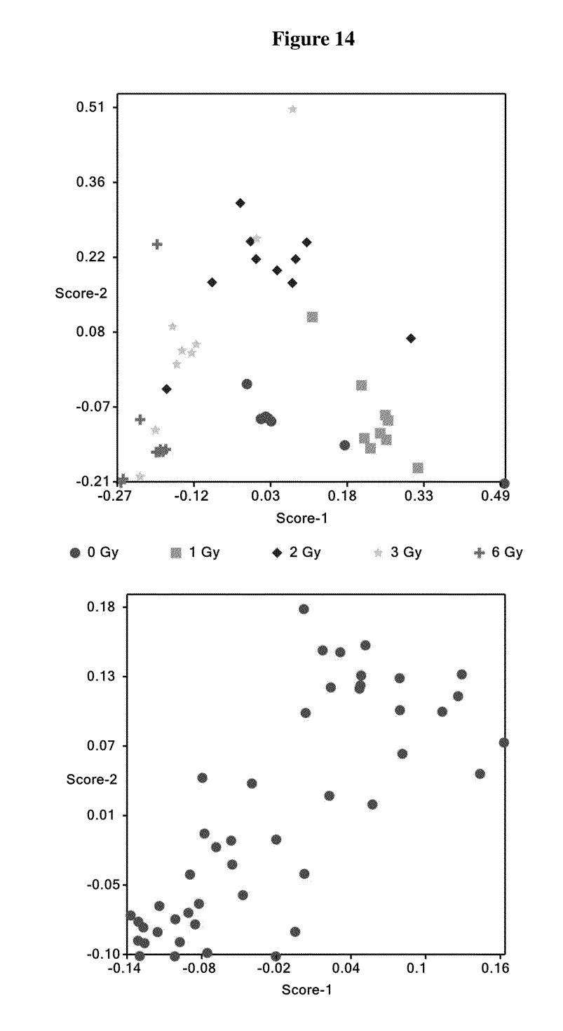

FIG. 14 shows the 2D score plots for supervised (top panel) and unsupervised (bottom panel) RF analysis of the proximity matrix based on biomarker response at day 1. For the supervised clustering representation shown in the top panel, good separation can be noticed between objects(mice) in the 0 Gy, 1 Gy, and 2 Gy classes, however, the 3 Gy objects are more scattered along with the 6 Gy objects. Unsupervised cluster representation of 2D scores (bottom panel) suggest that at most the cluster structure is comprised of 2 clusters of objects in the upper right and lower left of the score plot.

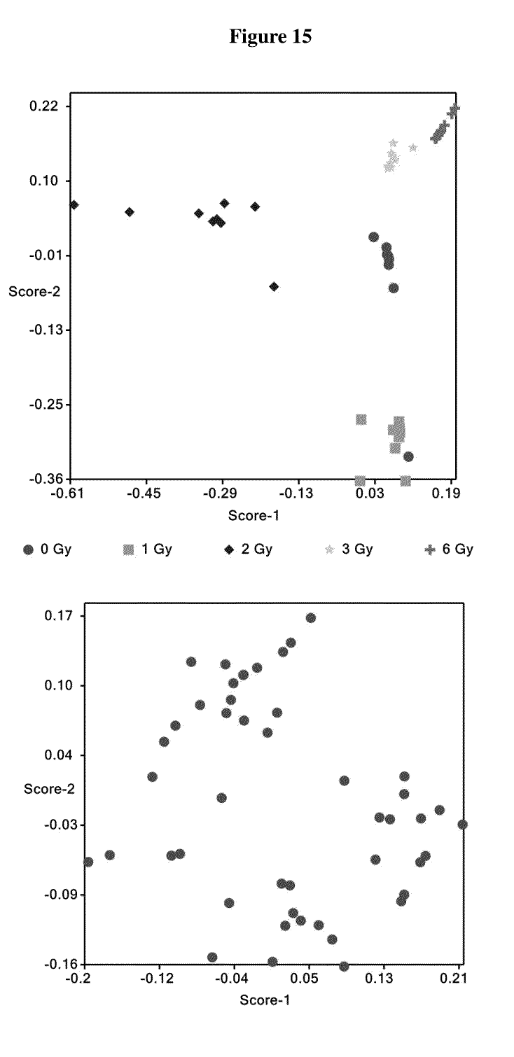

FIG. 15 presents the score plots for supervised (top panel) and unsupervised (bottom panel) cluster results based on day 5 biomarker responses. For the 5-day data, there is clearly marked separation between objects (mice) for supervised analysis, and the unsupervised results suggest a cluster structure having possibly 3 or 4 clusters.

Table 2 lists the 5-class accuracy for class prediction using the 8 supervised classifiers based on the most informative biomarkers for their day-1 response. The lowest mean accuracy was observed for SVMLS (49%) and the greatest mean accuracy occurred for PLOG (78%). The EMV mean accuracy was 80% however, and reflects the benefit of ensemble methods for combining multiple votes from a committee of classifiers. Classification accuracy using each method was (in decreasing order): EMV (80%), PLOG (78%), LDA (77%), 5NN (76%), CPSO (76%), EWMV (75%), LVQ1 (72%), ANN (71%), NBC (65%), and SVMLS (49%). Table 3 lists mean classification accuracy based on for day-5 biomarker responses used as inputs. The mean accuracy for NBC was quite low (53%), and mean accuracy for SVMLS (71%) and ANN (75%) were not markedly better. Classification accuracy using the various techniques was: LDA (100%), 5NN (100%), LVQ1 (100%), CPSO (100%), PLOG (98%), EMV (96%), EWMV (87%), ANN (75%), SVMLS (71%), and NBC (53%). The majority of classifiers showed outstanding performance for dose category prediction based on the 5-day biomarker responses.

Lastly, there was good agreement between the values of biomarker RF importance scores and biomarker filtering and selection results prior to input for supervised classification. The additional information provided by RF-based frequency of biomarker selection in first and all node splits reveals that, although a biomarker can suffer from having a low RF importance score, it can nevertheless be selected more often than other biomarkers with greater importance scores.

RF analysis allowed us to evaluate the informativeness of biomarker response at 1 and 5 days after exposure for the purpose of discriminating the 5 classes of dose. The advantage of RF lies in the strength of bootstrapping multiple realizations of the data and each time randomly selecting features for Gini evaluation for each node splitting in order to generate thousands of decision trees in a forest. RF's do not commonly overfit data and typically are the most reliable approach for generalizing resultsto future unobserved test data, which is mostly due to their conservativeness hinged to bootstrapping and random feature selection-evaluation during tree generation Importance scores are permutation-based and offer a means of evaluating feature informativeness based on null and alternative distributions, while the frequency of feature selection during first node splits and all node splits provides additional information regarding feature informativeness—because if a feature results in strong class separation, it will likely be identified via Gini in the first node split and numerous other splits within a decision tree. The supervised and unsupervised clustering results presented in 2D score plots of the proximity matrix reveal the cluster structureof objects based on training data with intact class labels and simulated class labels.

RF results based on all data indicate that gender and time since exposure are much less informative than the association between biomarker response and dose category. This is an impressive observation which suggests that in the context of all data, the four biomarkers evaluated are considerably more informative for predicting class when compared with mouse gender and time after exposure. For day 1 results, SAA1 and FLT3LG are almost equally informative when considering RF importance scores and supervised classification results. Filtering and selection methods for supervised classification resulted in SAA1 and FLT3LG being selected the majority of time during various filtration approaches. For day 5 biomarker response, FLT3LG dominated the importance scores, but VCAM, CC3 and SAA1 were nevertheless selected quite frequently during first and all node splits during decision tree generation. During feature filtration and selection, only FLT3LG was selected for the day 5 classification runs.

Feature selection approaches using various inferential hypothesis testing approaches did not result in markedly different classification performance. The NBC, SVMLS, and ANN supervised classifiers resulted in lower performance most likely because of the small number of features used. ANNs are biased toward the amount of training data used, tend to suffer when the feature number is low, and can also overfit the data if less than n˜200 objects are used per feature. For the day 1 data, the SVMLS suffered due to the increased overlap of mice in the 3 Gy and 6 Gy dose categories.

This study is a works-in-progress to develop classification methods for ELISA-based irradiation responses of proteomic biomarkers for dose category prediction. Gender and time-after-exposure dose-response curves, ROC curves, and ROC area under the curve for the biomarkers evaluated were not provided in this report since they form the basis of evaluations in our other reports.

Unsupervised class discovery and supervised class prediction of ionizing radiation dose category for mice exposed to 0 Gy, 1 Gy, 2 Gy, 3 Gy, and 6 Gy were investigated using plasma ELISA concentration of 4 proteomic biomarkers (FLT3LG, SAA1, CC3, and VCAM). Plasma ELISA concentrations were obtained in triplicate from n=50 mice at 1 day post-exposure and n=50 mice at 5 days post-exposure, with equal sample sizes of gender (male, female) at each dose level. Random forests (RF) were used for unsupervised analyses to evaluate biomarker informativeness, while 8 supervised classification techniques were employed for multiclass analysis of a 5-class problem (0,1,2,3,6 Gy). Classifiers included k nearest neighbor (kNN), naïve Bayes classifier (NBC), linear discriminant analysis (LDA), learning vector quantization (LVQ1), least squares support vector machines (SVMLS), artificial neural networks (ANN), constricted particle swarm optimization (CPSO), and polytomous logistic regression (PLOG). Results indicate that, for the biomarkers considered, gender and time since exposure were much less informative than the association between biomarker response and dose category. For day 1 results, SAA1 and FLT3LG are almost equally informative when considering RF importance scores and supervised classification results. For day 5 biomarker response, FLT3LG dominated the importance scores, but VCAM, CC3, and SAA1 were nevertheless selected quite frequently during first and all node splits during RF decision tree generation. During feature filtration and selection, only FLT3LG was selected for the day 5 classification runs. Feature selection approaches using various inferential hypothesis testing approaches did not result in markedly different classification performance. The RF analyses performed demonstrate that bootstrapping to generate alternative realizations of training data and simultaneous random selection of features during node splitting in decision tree learning is a superior approach to unsupervised and supervised classification analysis, especially for evaluating biomarker dose informativeness. Our findings lay the groundwork as additional radiation biomarkers become available to improve the cluster structure of the data and to improve supervised classification performance.

### REFERENCES

1. Marchetti, F., Coleman, M. A., Jones, I. M., Wyrobek, A. J. Candidate protein biodosimeters of human exposure to ionizing radiation. Int. J. Radiat. Biol. 82(9), 605-639, 2006.
2. Kim, D., Marchetti, F., Chen, Z., Zaric, S., Wilson, R. J., Hall, D. A., Gaster, R. S., Lee, J. R., Wang, J., Osterfeld, S. J., Yu, H., White, R. M., Blakely, W. F., Peterson, L. E., Bhatnagar, S., Mannion, B., Tseng, S., Roth, K., Coleman, M. A., Snijders, A. M., Wyrobek, A. J., Wang, S. X. Nanosensor dosimetry of mouse blood proteins after exposure to ionizing radiation. Scientific Reports(Nature). 3:2234, 2013.
3. Budworth, H., Snijders, A. M., Marchetti, F., Mannion, B., Bhatnagar, S., Kwoh, E., Tan, Y., Wang, S. X., Blakely, W. F., Coleman, M. A., Peterson, L. E., Wyrobek, A. J. DNA repair and cell cycle biomarkers of radiation exposure and inflammation stress in human blood. PLoS One. 7(11):e48619, 2012.
4. Breiman, L. Random Forests. Machine Learning. 45:5-32, 2001.
5. Peterson, L.E. Classification Analysis of DNA Microarrays. New York(NY), John Wiley and Sons, 2013.
6. Somol, P., Pudil, P., Nonovicova, J., and Paclik, J. Adaptive floating search methods in feature selection. Pattern Recognition Letters. 20:1157-1163, 1999
7. Fix, E., Hodges, J. L. Discriminatory analysis, non-parametric discrimination: Consistency properties. Technical Report 4, USAF School of Aviation Medicine, Randolph Field, Tex., 1951.
8. Kuncheva, L. I., Hoare, Z. S. Error-dependency relationships for the naive Bayes classifier with binary features. IEEE Trans. Pattern Anal. Mach. Intell. 30(4):735-740, 2008.
9. Fisher, R. A. The Use of Multiple Measurements in Taxonomic Problems. Annals of Eugenics. 7:179-188, 1936.
10. Flotzinger, D., Kalcher, J., Pfurtscheller, G. EEG classification by learning vector quantization. Biomed. Tech. (Berlin). 37(12):303-9, 1992.
11. Van Gestel, T., Suykens, J. A. K. Benchmarking least squares support vector machine classifiers. Machine Learning. 54, 5-32, 2004.
12. Leisch, F., Jain, L. C., Hornik, K. Cross-validation with active pattern selection for neural-network classifiers. IEEE Transactions on Neural Networks. 9:35-41, 1998.
13. Kennedy, J., Eberhart, R. C. Particle swarm optimization, Proceedings of IEEE International Conference on Neural Networks. Piscataway(NJ), 1995, 1942-1948. Hosmer, D.W., Lemeshow, S. Applied Logistic Regression. New York, John Wiley, 1989.
14. van Erp, M., Vuupijl, L., and Shomaker, L. An overview and comparison of voting methods for pattern recognition. Proc. 8th Int. Workshop Frontiers in Handwriting Recognition (WFHR02). Hoboken(NJ), IEEE Press, 2002
I. Glojnaric, Cuzic, S. Erakovic-Haber, V. Parnham, M. J. The serum amyloid A response to silver nitrate in mice and its inhibition by dexamethasone and macrolide antibiotics. Int. Immunopharmacol. 7:12 (2007).
T. Ogata, Yamazaki, H. Teshima, T. Kihara, A. Suzumoto, Y. Inoue, T. Nishimoto, N. Matsurra, N. Early administration of IL6-RA does not prevent radiation-induced lung injury in mice. Radiation Oncology. 5:26 (2010).
N. Ossetrova, Blakely, W. F. Multiple blood-proteins approach for early-response exposure assessment using an in vivo murine radiation model. Int. J. Radiat. Biol. 85:10, 837-850 (2009).
N. Ossetrova, Sandgren, D. J. Gallego, S. Blakely, W. F. Combined approach of hematological biomarkers and plasma protein SAA for improvement of radiation dose assessment triage in biodosimetry applications. Health Phys. 98, 204-208 (2010).
M. Prat, Demarquay, C. Frick, J. Dudoignon, N. Thierry, D. Bertho, J. M. Use of flt3 ligand to evaluate residual hematopoiesis after heterogeneous irradiation in mice. Radiat. Res. 166, 504-511 (2006).
M. Prat, Demarquay, C. Frick, J. Thierry, D. Gorin, N. C. Bertho, J. M. Radiation induced plasma Flt ligand concentration in mice: evidence for the implication of several cell types. Radiat. Res. 163, 408-417 (2005).
K. N. Rithidech, Honikel, L. Rieger, R. Xie, W. Fischer, T. Simon, S. R. Protein-expression profiles in mouse blood-plasma following acute whole-body exposure to (137)Cs gamma rays. Int. J. Radiat. Biol. 5, 432-447 (2009).
K. Sugimoto, Adachi, Y. Moriyama, K. Qiong, W. Nakayama, A. Hosono, M. Mori K. J. Induction of the expression of SCF in mouse by lethal irradiation. Growth Factors. 19, 219-231 (2001).

TABLE 1

| | All-possible-pairs | One-against-all | All-at-once |
|---|---|---|---|
| Test | (M = Ω(Ω − 1)/2) | (M = Ω) | M = 1 |
| T-test | Nₘ = #{j:pⱼ ≦ 0.05} | Nₘ = #{j:pⱼ ≦ 0.05} | — |
| Mann- | Nₘ = #{j:pⱼ ≦ 0.05} | Nₘ = #{j:pⱼ ≦ 0.05} | — |
| Whitney | | | |
| F-test | — | — | N = |
| | | | #{j:pⱼ ≦ 0.05} |
| Kruskal- | — | — | N = |
| Wallis | | | #{j:pⱼ ≦ 0.05} |
| Gini index | Nₘ = p/M | Nₘ = p/M | N = p |
| Entropy | Nₘ = p/M | Nₘ = p/M | N = p |

TABLE 2

| Selection method | Filter method | Test | 5NN | NBC | LDA | LVQ1 | SVMLS | ANN | CPSO | PLOG | EMV | EWMV | Selected features |
|---|---|---|---|---|---|---|---|---|---|---|---|---|---|
| Greedy PTA | Pairwise | T-Test | 0.80 | 0.67 | 0.80 | 0.72 | 0.59 | 0.74 | 0.80 | 0.83 | 0.83 | 0.76 | zlnsaa1, zlnflt31g |
| | | Mann-Whitney | 0.80 | 0.67 | 0.80 | 0.76 | 0.59 | 0.73 | 0.80 | 0.83 | 0.80 | 0.78 | zlnsaa1, zlnflt31g |
| | | Entropy | 0.80 | 0.67 | 0.80 | 0.76 | 0.59 | 0.70 | 0.80 | 0.83 | 0.83 | 0.80 | zlnsaa1, zlnflt31g |
| | | Gini | 0.80 | 0.67 | 0.80 | 0.83 | 0.59 | 0.67 | 0.80 | 0.83 | 0.85 | 0.80 | zlnsaa1, zlnflt31g |
| | One-against-all | T-Test | 0.80 | 0.67 | 0.80 | 0.70 | 0.22 | 0.73 | 0.80 | 0.83 | 0.80 | 0.76 | zlnsaa1, zlnflt31g |
| | | Mann-Whitney | 0.80 | 0.67 | 0.80 | 0.78 | 0.50 | 0.71 | 0.80 | 0.83 | 0.83 | 0.72 | zlnsaa1, zlnflt31g |
| | | Entropy | 0.80 | 0.67 | 0.80 | 0.78 | 0.50 | 0.70 | 0.80 | 0.83 | 0.83 | 0.72 | zlnsaa1, zlnflt31g |
| | | Gini | 0.80 | 0.67 | 0.80 | 0.76 | 0.48 | 0.71 | 0.80 | 0.83 | 0.83 | 0.72 | zlnsaa1, zlnflt31g |
| | All-at-once | F-Test | 0.80 | 0.67 | 0.80 | 0.80 | 0.22 | 0.72 | 0.80 | 0.83 | 0.85 | 0.78 | zlnsaa1, zlnflt31g |
| | | Kruskal-Wallis | 0.80 | 0.67 | 0.80 | 0.78 | 0.50 | 0.67 | 0.80 | 0.83 | 0.83 | 0.76 | zlnflt31g, zlnsaa1 |
| | | Entropy | 0.80 | 0.67 | 0.80 | 0.76 | 0.50 | 0.72 | 0.80 | 0.83 | 0.83 | 0.76 | zlnflt31g, zlnsaa1 |
| | | Gini | 0.80 | 0.67 | 0.80 | 0.74 | 0.48 | 0.70 | 0.80 | 0.83 | 0.83 | 0.78 | zlnflt31g, zlnsaa1 |
| Best ranked N | Pairwise | T-Test | 0.80 | 0.67 | 0.80 | 0.78 | 0.59 | 0.72 | 0.80 | 0.83 | 0.85 | 0.76 | zlnsaa1, zlnflt31g |
| | | Mann-Whitney | 0.80 | 0.67 | 0.80 | 0.78 | 0.33 | 0.73 | 0.80 | 0.83 | 0.83 | 0.76 | zlnsaa1, zlnflt31g |
| | | Entropy | 0.48 | 0.48 | 0.54 | 0.43 | 0.54 | 0.73 | 0.46 | 0.46 | 0.61 | 0.72 | zlnsaa1, zlncc3 |
| | | Gini | 0.48 | 0.48 | 0.54 | 0.41 | 0.54 | 0.72 | 0.48 | 0.46 | 0.59 | 0.74 | zlnsaa1, zlncc3 |
| | One-against-all | T-Test | 0.80 | 0.67 | 0.80 | 0.76 | 0.50 | 0.70 | 0.80 | 0.83 | 0.85 | 0.74 | zlnsaa1, zlnflt31g |
| | | Mann-Whitney | 0.48 | 0.48 | 0.54 | 0.46 | 0.43 | 0.71 | 0.48 | 0.46 | 0.61 | 0.72 | zlnsaa1, zlncc3 |
| | | Entropy | 0.80 | 0.67 | 0.80 | 0.74 | 0.50 | 0.68 | 0.80 | 0.83 | 0.80 | 0.76 | zlnsaa1, zlnflt31g |
| | | Gini | 0.80 | 0.67 | 0.80 | 0.78 | 0.48 | 0.70 | 0.80 | 0.83 | 0.85 | 0.78 | zlnsaa1, zlnflt31g |
| | All-at-once | F-Test | 0.80 | 0.67 | 0.80 | 0.78 | 0.50 | 0.66 | 0.80 | 0.83 | 0.83 | 0.74 | zlnsaa1, zlnflt31g |
| | | Kruskal-Wallis | 0.80 | 0.67 | 0.80 | 0.74 | 0.50 | 0.74 | 0.80 | 0.83 | 0.80 | 0.74 | zlnflt31g, zlnsaa1 |
| | | Entropy | 0.80 | 0.67 | 0.80 | 0.76 | 0.48 | 0.74 | 0.80 | 0.83 | 0.83 | 0.78 | zlnflt31g, zlnsaa1 |
| | | Gini | 0.80 | 0.67 | 0.80 | 0.76 | 0.50 | 0.71 | 0.80 | 0.83 | 0.78 | 0.72 | zlnflt31g, zlnsaa1 |
| | | Average | 0.76 | 0.65 | 0.77 | 0.72 | 0.49 | 0.71 | 0.76 | 0.78 | 0.80 | 0.75 | |

TABLE 3

| | | | | | | | | | | | | | Selected |
|---|---|---|---|---|---|---|---|---|---|---|---|---|---|
| Selection method | Filter method | Test | 5NN | NBC | LDA | LVQ1 | SVMLS | ANN | CPSO | PLOG | EMV | EWMV | feature |
| Greedy PTA | Pairwise | T-Test | 1.00 | 0.53 | 1.00 | 1.00 | 0.98 | 0.74 | 1.00 | 0.98 | 0.98 | 0.98 | zlnflt31g |
| | | Mann-Whitney test | 1.00 | 0.53 | 1.00 | 1.00 | 1.00 | 0.77 | 1.00 | 0.98 | 1.00 | 0.98 | zlnflt31g |
| | | Entropy | 1.00 | 0.53 | 1.00 | 1.00 | 1.00 | 0.74 | 1.00 | 0.98 | 1.00 | 1.00 | zlnflt31g |
| | | Gini | 1.00 | 0.53 | 1.00 | 1.00 | 1.00 | 0.74 | 1.00 | 0.98 | 1.00 | 0.96 | zlnflt31g |
| | One-against-all | T-Test | 1.00 | 0.53 | 1.00 | 1.00 | 0.40 | 0.76 | 1.00 | 0.98 | 1.00 | 0.89 | zlnflt31g |
| | | Mann-Whitney test | 1.00 | 0.53 | 1.00 | 1.00 | 0.89 | 0.71 | 1.00 | 0.98 | 0.94 | 0.94 | zlnflt31g |
| | | Entropy | 1.00 | 0.53 | 1.00 | 1.00 | 0.87 | 0.73 | 1.00 | 0.98 | 0.94 | 0.94 | zlnflt31g |
| | | Gini | 1.00 | 0.53 | 1.00 | 1.00 | 0.21 | 0.73 | 1.00 | 0.98 | 0.94 | 0.91 | zlnflt31g |
| | All-at-once | F-Test | 1.00 | 0.53 | 1.00 | 1.00 | 0.21 | 0.73 | 1.00 | 0.98 | 0.96 | 0.89 | zlnflt31g |
| | | Kruskal-Wallis | 1.00 | 0.53 | 1.00 | 1.00 | 0.89 | 0.80 | 1.00 | 0.98 | 0.96 | 0.96 | zlnflt31g |
| | | Entropy | 1.00 | 0.53 | 1.00 | 1.00 | 0.21 | 0.77 | 1.00 | 0.98 | 0.94 | 0.89 | zlnflt31g |
| | | Gini | 1.00 | 0.53 | 1.00 | 1.00 | 0.87 | 0.71 | 1.00 | 0.98 | 0.96 | 0.96 | zlnflt31g |
| Best ranked N | Pairwise | T-Test | 1.00 | 0.53 | 1.00 | 1.00 | 1.00 | 0.76 | 1.00 | 0.98 | 1.00 | 0.98 | zlnflt31g |
| | | Mann-Whitney test | 1.00 | 0.53 | 1.00 | 1.00 | 0.89 | 0.76 | 1.00 | 0.98 | 0.96 | 0.91 | zlnflt31g |
| | | Entropy | 1.00 | 0.53 | 1.00 | 1.00 | 1.00 | 0.76 | 1.00 | 0.98 | 1.00 | 1.00 | zlnflt31g |
| | | Gini | 1.00 | 0.53 | 1.00 | 1.00 | 0.38 | 0.73 | 1.00 | 0.98 | 0.85 | 0.94 | zlnflt31g |
| | One-against-all | T-Test | 1.00 | 0.53 | 1.00 | 1.00 | 0.21 | 0.74 | 1.00 | 0.98 | 0.96 | 0.98 | zlnflt31g |
| | | Mann-Whitney test | 1.00 | 0.53 | 1.00 | 1.00 | 0.21 | 0.76 | 1.00 | 0.98 | 0.91 | 0.89 | zlnflt31g |
| | | Entropy | 1.00 | 0.53 | 1.00 | 1.00 | 0.96 | 0.78 | 1.00 | 0.98 | 0.98 | 0.94 | zlnflt31g |
| | | Gini | 1.00 | 0.53 | 1.00 | 1.00 | 0.89 | 0.76 | 1.00 | 0.98 | 0.94 | 0.94 | zlnflt31g |
| | All-at-once | F-Test | 1.00 | 0.53 | 1.00 | 1.00 | 0.96 | 0.80 | 1.00 | 0.98 | 0.98 | 0.94 | zlnflt31g |
| | | Kruskal-Wallis | 1.00 | 0.53 | 1.00 | 1.00 | 0.21 | 0.77 | 1.00 | 0.98 | 0.91 | 0.91 | zlnflt31g |
| | | Entropy | 1.00 | 0.53 | 1.00 | 1.00 | 0.89 | 0.77 | 1.00 | 0.98 | 0.96 | 0.94 | zlnflt31g |
| | | Gini | 1.00 | 0.53 | 1.00 | 1.00 | 0.87 | 0.72 | 1.00 | 0.98 | 0.94 | 0.94 | zlnflt31g |
| | | Average | 1.00 | 0.53 | 1.00 | 1.00 | 0.71 | 0.75 | 1.00 | 0.98 | 0.96 | 0.87 | |

TABLE 4

| | 24 h | | | 5 d | | |
|---|---|---|---|---|---|---|
| | Average | Average | Average | Average | Average | Average Concentration |
| A. Flt-31g | Concentration ± | Concentration for | Concentration for | Concentration | Concentration for | for Females ± |
| Dose (Gy) | SD (pg/ml) | Males ± SD (pg/ml) | Females ± SD (pg/ml) | (pg/ml) | Males ± SD (pg/ml) | SD (pg/ml) |
| 0 | 294 ± 53 | 281.63 ± 65.3 | 306.5 ± 29.3 | 255.93 ± 21.8 | 243.8 ± 18.0 | 276.07 ± 9.2 |
| 1 | 445.1* ± 48 | 421.246* ± 29.1 | 468.9* ± 49.1 | 419.1* ± 43.1 | 406.3* ± 29.8 | 431.9* ± 50.0 |
| 2 | 596.4* ± 83 | 567.9* ± 62.7 | 625.0* ± 88.9 | 652.7* ± 82 | 639.2* ± 83.0 | 669.5* ± 75.0 |
| 3 | 724.2* ± 94 | 761.9* ± 108.5 | 686.3* ± 51.32 | 1427.2* ± 255.3 | 1650.5* ± 167.9 | 1203.9* ± 49.6 |
| 6 | 912.9* ± 126 | 853.9* ± 109.7 | 971.9* ± 108.8 | 3746.9* ± 779 | 3359.2* ± 592.5 | 4134.5* ± 721.8 |
| | 24 h | | | 5 d | | |
| | Average | Average | Average | Average | Average | Average Concentration |
| B. SAA1 | Concentration ± | Concentration for | Concentration for | Concentration ± SD | Concentration for | for Females ± |
| Dose (Gy) | SD (μg/ml) | Males ± SD (μg/ml) | Females ± SD (μg/ml) | (μg/ml) | Males ± SD (μg/ml) | SD (μg/ml) |
| 0 | 99.7 ± 19.9 | 98.7 ± 15.3 | 100.9 ± 23.6 | 69.5 ± 10.1 | 69.126 ± 4.9 | 69.9 ± 13.4 |
| 1 | 250.9* ± 87.9 | 304.4* ± 89.0 | 197.4* ± 42.5 | 73.4 ± 8.8 | 74.2* ± 9.9 | 72.7 ± 7.4 |
| 2 | 617* ± 119 | 629.2* ± 65.6 | 574.3* ± 125.4 | 70.8 ± 21.3 | 71.7 ± 24.5 | 69.9 ± 17.6 |
| 3 | 655* ± 147 | 669.2* ± 140.5 | 640.8* ± 147.1 | 71 ± 13.2 | 63.9 ± 13.2 | 78.2* ± 8.4 |
| 6 | 695.4* ± 151.3 | 723.9* ± 163.8 | 667.0* ± 124.8 | 65.3 ± 9.7 | 65.3 ± 11.7 | 65.3 ± 8.2 |
| | 24 h | | | 5 d | | |
| | Average | Average | Average | Average | Average | Average Concentration |
| C. CC3 | Concentration ± | Concentration for | Concentration for | Concentration | Concentration for | for Females ± |
| Dose (Gy) | SD (μg/ml) | Males ± SD (μg/ml) | Females ± SD (μg/ml) | (μg/ml) | Males ± SD (μg/ml) | SD (μg/ml) |
| 0 | 680 ± 109 | 616.755 ± 123.7 | 642.838 ± 137.4 | 592.3 ± 145.6 | 484.4 ± 44.2 | 700.4 ± 132.7 |
| 1 | 773* ± 138 | 794.434* ± 97.7 | 722.485 ± 144.2 | 668.3 ± 157.2 | 568.9 ± 111.7 | 768.01 ± 125.3 |
| 2 | 849* ± 169 | 809.5* ± 150.3 | 862.2* ± 195.0 | 607.7 ± 121 | 528.3 ± 90.8 | 687.7 ± 87.9 |
| 3 | 1094* ± 256 | 946.3* ± 193.8 | 1136.2* ± 183.7 | 662.8 ± 157 | 529.6 ± 50.9 | 796.7 ± 99.0 |
| 6 | 981* ± 204 | 863.7* ± 131.9 | 1099.6* ± 187.9 | 787 ± 196 | 813.3* ± 231.4 | 748.4 ± 133.9 |
| | 24 h | | | 5 d | | |
| | Average | Average | Average | Average | Average | Average Concentration |
| D. VCAM1 | Concentration ± | Concentration for | Concentration for | Concentration | Concentration for | for Females ± |
| Dose (Gy) | SD (μg/ml) | Males ± SD (μg/ml) | Females ± SD (μg/ml) | (pg/ml) | Males ± SD (μg/ml) | SD (μg/ml) |
| 0 | 772 ± 228.3 | 856.1 ± 255.0 | 689.2 ± 147.3 | 883.3 ± 236.7 | 887.5 ± 280.9 | 879.468 ± 174.2 |
| 1 | 686.5 ± 183.6 | 742.5 ± 215.7 | 639.4 ± 124.8 | 771.8 ± 224.07 | 896.9 ± 223.4 | 650.8* ± 135.8 |
| 2 | 633.4 ± 180.0 | 692.5* ± 184.6 | 578.5* ± 155.6 | 712.2* ± 219.1 | 644.6* ± 244.2 | 780.7 ± 156.7 |
| 3 | 620.9* ± 106.8 | 644.7* ± 114.5 | 599.4* ± 93.12 | 691.6* ± 170.2 | 751.8 ± 184.1 | 633.7* ± 122.8 |
| 6 | 566.7* ± 129.4 | 620.8* ± 110.5 | 513.1* ± 120.2 | 436.3* ± 142.5 | 368.0* ± 128.7 | 505.4* ± 118.6 |

*indicates significance difference

TABLE 5
Day 1 data
7NN, LOOCV

| Dose(Gy) | FLT3LG | SAA1 | CC3 | VCAM | MULTI |
|---|---|---|---|---|---|
| 0 vs. 1 | 0.97 | 0.98 | 0.74 | 0.40 | 1.00 |
| 0 vs. 2 | 1.00 | 1.00 | 0.78 | 0.44 | 1.00 |
| 0 vs. 3 | 1.00 | 1.00 | 0.95 | 0.71 | 1.00 |
| 0 vs. 6 | 1.00 | 1.00 | 0.92 | 0.74 | 1.00 |
| 1 vs. 2 | 0.96 | 1.00 | 0.71 | 0.42 | 0.98 |
| 1 vs. 3 | 1.00 | 1.00 | 0.82 | 0.59 | 1.00 |
| 1 vs. 6 | 1.00 | 1.00 | 0.75 | 0.65 | 1.00 |
| 2 vs. 3 | 0.80 | 0.61 | 0.86 | 0.64 | 0.85 |
| 2 vs. 6 | 0.98 | 0.73 | 0.75 | 0.62 | 0.97 |
| 3 vs. 6 | 0.88 | 0.76 | 0.46 | 0.64 | 0.77 |
| Avg | 0.96 | 0.91 | 0.77 | 0.59 | 0.96 |

Day 1 data
LDA, LOOCV

| Dose(Gy) | FLT3LG | SAA1 | CC3 | VCAM | MULTI |
|---|---|---|---|---|---|
| 0 vs. 1 | 0.98 | 0.99 | 0.73 | 0.56 | 1.00 |
| 0 vs. 2 | 1.00 | 1.00 | 0.80 | 0.66 | 1.00 |
| 0 vs. 3 | 1.00 | 1.00 | 0.96 | 0.68 | 1.00 |
| 0 vs. 6 | 1.00 | 1.00 | 0.93 | 0.77 | 1.00 |
| 1 vs. 2 | 0.97 | 1.00 | 0.61 | 0.55 | 1.00 |
| 1 vs. 3 | 1.00 | 1.00 | 0.84 | 0.58 | 1.00 |
| 1 vs. 6 | 1.00 | 1.00 | 0.78 | 0.68 | 1.00 |
| 2 vs. 3 | 0.85 | 0.57 | 0.76 | 0.09 | 0.87 |
| 2 vs. 6 | 0.98 | 0.66 | 0.66 | 0.57 | 0.98 |
| 3 vs. 6 | 0.87 | 0.51 | 0.59 | 0.59 | 0.90 |
| Avg | 0.97 | 0.87 | 0.77 | 0.57 | 0.98 |

Day 5 data
7NN, LOOCV

| Dose(Gy) | FLT3LG | SAA1 | CC3 | VCAM | MULTI |
|---|---|---|---|---|---|
| 0 vs. 1 | 1.00 | 0.70 | 0.68 | 0.62 | 0.99 |
| 0 vs. 2 | 1.00 | 0.79 | 0.67 | 0.69 | 1.00 |
| 0 vs. 3 | 1.00 | 0.74 | 0.62 | 0.74 | 1.00 |
| 0 vs. 6 | 1.00 | 0.83 | 0.87 | 0.92 | 1.00 |
| 1 vs. 2 | 1.00 | 0.78 | 0.54 | 0.52 | 0.98 |
| 1 vs. 3 | 1.00 | 0.57 | 0.58 | 0.64 | 1.00 |
| 1 vs. 6 | 1.00 | 0.83 | 0.61 | 0.91 | 1.00 |
| 2 vs. 3 | 1.00 | 0.71 | 0.59 | 0.58 | 1.00 |
| 2 vs. 6 | 1.00 | 0.71 | 0.72 | 0.80 | 1.00 |
| 3 vs. 6 | 1.00 | 0.79 | 0.68 | 0.81 | 1.00 |
| Avg | 1.00 | 0.75 | 0.66 | 0.72 | 1.00 |

Day 5 data
LDA, LOOCV

| Dose(Gy) | FLT3LG | SAA1 | CC3 | VCAM | MULTI |
|---|---|---|---|---|---|
| 0 vs. 1 | 1.00 | 0.60 | 0.59 | 0.66 | 1.00 |
| 0 vs. 2 | 1.00 | 0.00 | 0.49 | 0.68 | 1.00 |
| 0 vs. 3 | 1.00 | 0.50 | 0.60 | 0.74 | 1.00 |
| 0 vs. 6 | 1.00 | 0.46 | 0.79 | 0.96 | 1.00 |
| 1 vs. 2 | 1.00 | 0.52 | 0.58 | 0.54 | 1.00 |
| 1 vs. 3 | 1.00 | 0.52 | 0.20 | 0.59 | 1.00 |
| 1 vs. 6 | 1.00 | 0.41 | 0.68 | 0.92 | 1.00 |
| 2 vs. 3 | 1.00 | 0.46 | 0.55 | 0.30 | 1.00 |
| 2 vs. 6 | 1.00 | 0.52 | 0.77 | 0.84 | 1.00 |
| 3 vs. 6 | 1.00 | 0.47 | 0.70 | 0.87 | 1.00 |
| Avg | 1.00 | 0.45 | 0.60 | 0.71 | 1.00 |

TABLE 6
Day 1
7NN, LOOCV

| Dose(Gy) | FLT3LG | SAA1 | CC3 | VCAM | MULTI |
|---|---|---|---|---|---|
| 0 vs. other | 0.99 | 0.98 | 0.84 | 0.70 | 1.00 |
| 1 vs. other | 0.97 | 0.99 | 0.69 | 0.52 | 0.99 |
| 2 vs. other | 0.87 | 0.80 | 0.78 | 0.52 | 0.92 |
| 3 vs. other | 0.86 | 0.82 | 0.78 | 0.66 | 0.88 |
| 6 vs. other | 0.96 | 0.86 | 0.60 | 0.72 | 0.95 |
| Avg | 0.93 | 0.89 | 0.74 | 0.62 | 0.95 |

Day 1
LDA, LOOCV

| Dose(Gy) | FLT3LG | SAA1 | CC3 | VCAM | MULTI |
|---|---|---|---|---|---|
| 0 vs. other | 1.00 | 1.00 | 0.86 | 0.68 | 1.00 |
| 1 vs. other | 0.78 | 0.75 | 0.62 | 0.55 | 0.70 |
| 2 vs. other | 0.50 | 0.67 | 0.33 | 0.51 | 0.83 |
| 3 vs. other | 0.73 | 0.76 | 0.79 | 0.52 | 0.80 |
| 6 vs. other | 0.96 | 0.79 | 0.68 | 0.66 | 0.94 |
| Avg | 0.79 | 0.79 | 0.66 | 0.58 | 0.85 |

Day 5
7NN, LOOCV

| Dose(Gy) | FLT3LG | SAA1 | CC3 | VCAM | MULTI |
|---|---|---|---|---|---|
| 0 vs. other | 1.00 | 0.81 | 0.68 | 0.74 | 1.00 |
| 1 vs. other | 1.00 | 0.72 | 0.59 | 0.62 | 0.99 |
| 2 vs. other | 1.00 | 0.66 | 0.58 | 0.55 | 0.99 |
| 3 vs. other | 1.00 | 0.66 | 0.66 | 0.67 | 1.00 |
| 6 vs. other | 1.00 | 0.84 | 0.79 | 0.87 | 1.00 |
| Avg | 1.00 | 0.74 | 0.66 | 0.69 | 1.00 |

Day 5
LDA, LOOCV

| Dose(Gy) | FLT3LG | SAA1 | CC3 | VCAM | MULTI |
|---|---|---|---|---|---|
| 0 vs. other | 1.00 | 0.53 | 0.64 | 0.77 | 0.99 |
| 1 vs. other | 0.78 | 0.32 | 0.00 | 0.60 | 0.74 |
| 2 vs. other | 0.53 | 0.51 | 0.59 | 0.47 | 0.47 |
| 3 vs. other | 0.73 | 0.48 | 0.33 | 0.41 | 0.78 |
| 6 vs. other | 1.00 | 0.46 | 0.74 | 0.90 | 1.00 |
| Avg | 0.81 | 0.46 | 0.46 | 0.63 | 0.80 |

TABLE 7
A.

| Dose | 0 Gy | 1 Gy | 2 Gy | 3 Gy | 6 Gy |
|---|---|---|---|---|---|
| 0 Gy | | −5.0462 | −7.3541 | −8.8049 | −10.3500 |
| | | (p = 0.0001) | (p < 0.0001) | (p < 0.0001) | (p < 0.0001) |
| 1 Gy | −8.5913 | | −5.5186 | −9.1781 | −13.0835 |
| | (p < 0.0001) | | (p < 0.0001) | (p < 0.0001) | (p < 0.0001) |
| 2 Gy | −12.8952 | −8.0332 | | −3.4246 | −7.2693 |
| | (p < 0.0001) | (p < 0.0001) | | (p < 0.003) | (p < 0.0001) |
| 3 Gy | −19.5497 | −18.5101 | −10.8030 | | −3.9905 |
| | (P < 0.0001) | (p < 0.0001) | (p < 0.0001) | | (p = 0.0009) |
| 6 Gy | −26.1994 | −29.4066 | −21.8601 | −11.0419 | |
| | (p < 0.0001) | (p < 0.0001) | (p < 0.0001) | (p < 0.0001) | |

B.

| | 0 Gy | | 1 Gy | | 2 Gy |
|---|---|---|---|---|---|
| 1 Gy, 2 Gy | −7.3546 | 2 Gy, 3 Gy | −7.2602 | 3Gy, 6Gy | −5.1277 |
| | (p < 0.0001) | | (p < 0.0001) | | (p < 0.0001) |
| 1 Gy, 2 Gy, 3 Gy | −8.5374 | 2 Gy, 3 Gy, | −7.5103 | | |
| | (p < 0.0001) | 6 Gy | (p < 0.0001) | | |
| 1 Gy, 2 Gy, | −8.9112 | | | | |
| 3 Gy, 6 Gy | (p < 0.0001) | | | | |

C.

| | 0 Gy | | 1 Gy | | 2 Gy |
|---|---|---|---|---|---|
| 1 Gy, 2 Gy | −9.3746 | 2 Gy, 3 Gy | −6.8872 | 3 Gy, 6 Gy | −7.5585 |
| | (p < 0.0001) | | (p < 0.0001) | | (p < 0.0001) |
| 1 Gy, 2 Gy, 3 Gy | −7.6093 | 2 Gy, 3 Gy, | −6.2978 | | |
| | (p < 0.0001) | 6 Gy | (p < 0.0001) | | |
| 1 Gy, 2 Gy, | −6.6695 | | | | |
| 3 Gy, 6 Gy | (p < 0.0001) | | | | |

TABLE 8
A.

| Dose | 0 Gy | 1 Gy | 2 Gy | 3 Gy | 6 Gy |
|---|---|---|---|---|---|
| 0 Gy | | −7.4755 | −21.2084 | −19.7527 | −21.9760 |
| | | (p < 0.0001) | (p < 0.0001) | (p < 0.0001) | (p < 0.0001) |
| 1 Gy | −1.1790 | | −6.9865 | −7.1083 | −7.8703 |
| | (p = 0.2537) | | (p < 0.0001) | (p < 0.0001) | (p < 0.0001) |
| 2 Gy | 0.8380 | 1.2784 | | −0.7765 | −1.6285 |
| | (p = 0.4130) | (p = 0.2173) | | (p = 0.4476) | (p = 0.1208) |
| 3 Gy | 0.0403 | 0.9274 | −0.7834 | | −0.6685 |
| | (P = 0.9683) | (p = 0.3660) | (p = 0.4436) | | (p = 0.5123) |
| 6 Gy | −1.1724 | −0.8918 | −1.4929 | −1.1589 | |
| | (p = 0.2563) | (p = 0.3843 | (p = 0.1528) | (p = 0.2616) | |

B.

| | 0 Gy | | 1 Gy | | 2 Gy |
|---|---|---|---|---|---|
| 1 Gy, 2 Gy | −8.2570 | 2 Gy, 3 Gy | −9.1331 | 3 Gy, 6 Gy | −1.3347 |
| | (p < 0.0001) | | (p < 0.0001) | | p = 0.1927 |
| 1 Gy, 2 Gy, 3 Gy | −9.7344 | 2 Gy, 3 Gy, | −10.7110 | | |
| | (p < 0.0001) | 6 Gy | (p < 0.0001) | | |
| 1 Gy, 2 Gy, | −11.0084 | | | | |
| 3 Gy, 6 Gy | (p < 0.0001) | | | | |

C.

| | 0 Gy | | 1 Gy | | 2 Gy |
|---|---|---|---|---|---|
| 1 Gy, 2 Gy | 0.3071 | 2 Gy, 3 Gy | 1.1058 | 3 Gy, 6 Gy | −1.3643 |
| | (p = 0.7611) | | (p = 0.2782) | | (p = 0.1833) |
| 1 Gy, 2 Gy, 3 Gy | 0.2452 | 2 Gy, 3 Gy, | 0.1742 | | |
| | (p = 0.8076) | 6 Gy | (p = 0.8626) | | |
| 1 Gy, 2 Gy, | −0.3083 | | | | |
| 3 Gy, 6 Gy | (p = 0.7592) | | | | |

TABLE 9
A.

| Dose | 0 Gy | 1 Gy | 2 Gy | 3 Gy | 6 Gy |
|---|---|---|---|---|---|
| 0 Gy | | −1.8510 | −2.5038 | −4.9000 | −4.4508 |
| | | (p = 0.0827) | (p = 0.0228) | (p = 0.0002) | (p = 0.0004) |
| 1 Gy | −1.1609 | | −0.7456 | −3.2632 | −2.6092 |
| | (p = 0.2608) | | (p = 0.4661) | (p = 0.0049) | (p = 0.0183) |
| 2 Gy | −0.4103 | −2.7070 | | −2.3598 | −1.6532 |
| | (p = 0.6864) | (p = 0.0144) | | (p = 0.0305) | (p = 0.1156) |
| 3 Gy | −1.1125 | 0.8382 | −0.7805 | | 0.9893 |
| | (P = 0.2805) | (p = 0.4129) | (p = 0.4452) | | (p = 0.3364) |
| 6 Gy | −2.7070 | 0.0715 | −2.5252 | −1.8606 | |
| | (p = 0.0144) | (p = 0.9438) | (p = 0.0212) | (p = 0.0792) | |

B.

| | 0 Gy | | 1 Gy | | 2 Gy |
|---|---|---|---|---|---|
| 1 Gy, 2 Gy | −2.5881 | 2 Gy, 3 Gy | −1.9910 | 3 Gy, 6 Gy | −2.4292 |
| | (p = 0.0156) | | (p = 0.0571) | | (p = 0.0221) |
| 1 Gy, 2 Gy, 3 Gy | −3.4681 | 2 Gy, 3 Gy, | −2.4416 | | |
| | (p = 0.0014) | 6 Gy | (p = 0.0197) | | |
| 1 Gy, 2 Gy, | −4.0786 | | | | |
| 3 Gy, 6 Gy | (p = 0.0002) | | | | |

C.

| | 0 Gy | | 1 Gy | | 2 Gy |
|---|---|---|---|---|---|
| 1 Gy, 2 Gy | −0.9422 | 2 Gy, 3 Gy | 0.5291 | 3 Gy, 6 Gy | −1.8289 |
| | (p = 0.3542) | | (p = 0.6009) | | (p = 0.0781) |
| 1 Gy, 2 Gy, 3 Gy | −1.1570 | 2 Gy, 3 Gy, | −0.4424 | | |
| | (p = 0.2545) | 6 Gy | (p = 0.6607) | | |
| 1 Gy, 2 Gy, | −1.7030 | | | | |
| 3 Gy, 6 Gy | (p = 0.0950) | | | | |

TABLE 10
A.

| Dose | 0 Gy | 1 Gy | 2 Gy | 3 Gy | 6 Gy |
|---|---|---|---|---|---|
| 0 Gy | | 1.2190 | 1.8135 | 2.3248 | 3.0527 |
| | | (p = 0.2386) | (p = 0.0865) | (p = 0.0320) | (p = 0.0069) |
| 1 Gy | 1.3358 | | 0.9511 | 1.4306 | 2.4058 |
| | (p = 0.1983) | | (p = 0.3541) | (p = 0.1697) | (p = 0.0271) |
| 2 Gy | 1.8468 | 0.7582 | | 0.0488 | 0.3198 |
| | (p = 0.0813) | (p = 0.4581) | | (p = 0.9616) | (p = 0.1156) |
| 3 Gy | 2.4546 | 1.1292 | 0.1315 | | 1.3080 |
| | (P = 0.0245) | (p = 0.2737) | (p = 0.8968) | | (p = 0.2073) |
| 6 Gy | 6.1192 | 5.1143 | 3.5854 | 4.2832 | |
| | (p < 0.0001) | (p = 0.0001) | (p = 0.0021) | (p = 0.0004) | |

B.

| | 0 Gy | | 1 Gy | | 2 Gy |
|---|---|---|---|---|---|
| 1 Gy, 2 Gy | 1.8303 | 2 Gy, 3 Gy | 1.2782 | 3 Gy, 6 Gy | 0.6928 |
| | (p = 0.0779) | | (p = 0.2117) | | (p = 0.4942) |
| 1 Gy, 2 Gy, 3 Gy | 2.3487 | 2 Gy, 3 Gy, 6 Gy | 1.8001 | | |
| | (p = 0.0241) | | (p = 0.0798) | | |
| 1 Gy, 2 Gy, | 2.8315 | | | | |
| 3 Gy, 6 Gy | (p = 0.0067) | | | | |

C.

| | 0 Gy | | 1 Gy | | 2 Gy |
|---|---|---|---|---|---|
| 1 Gy, 2 Gy | 1.8422 | 2 Gy, 3 Gy | 1.0469 | 3 Gy, 6 Gy | 1.9279 |
| | (p = 0.0761) | | (0.3041) | | (p = 0.0641) |
| 1 Gy, 2 Gy, 3 Gy | 2.3013 | 2 Gy, 3 Gy, 6 Gy | 2.2108 | | |
| | (p = 0.0269) | | (p = 0.0331) | | |
| 1 Gy, 2 Gy, | 2.9783 | | | | |
| 3 Gy, 6 Gy | (p = 0.0045) | | | | |

What is claimed is:

1. A panel of genetic probes for determining radiation dosage classification of a patient, said panel comprising genetic probes to detect the genes or gene products of FLT3LG, SAA1, CC3, and/or VCAM-1, and combinations thereof.

2. A method for determining the radiation dosage received by a patient, comprising the steps of: (a) obtaining a blood sample from a patient; (b) conducting an enzyme-linked immunosorbant assay (ELISA) on the sample; (c) determining the FLT3LG, SAA1, CC3, and/or VCAM-1 protein concentrations in the patient blood sample; (d) standardizing the protein concentration amounts for each protein; (e) transforming said protein concentration amounts through the classification algorithms to produce prediction scores for the dose classification of the patient sample, and (f) determining the radiation dosage of the patient sample based on the prediction score..

3. The method of claim 1, wherein said protein concentration is determined by optical density.

4. The method of claim 1 further comprising: (g) triaging the patient according to the severity of the dosage received and then appropriate treatments are recommended and prescribed.

5. A panel of genetic probes for determining radiation exposure in a patient, said panel comprising genetic probes to detect the genes or gene products of FLT3LG, SAA1, CC3 and VCAM-1.
